(19) United States
(12) Patent Application Publication
(10) Pub. No.: US 2026/0091476 A1
(43) Pub. Date: Apr. 2, 2026

(54) SMART HAND-HELD POWER TOOL SYSTEM WITH SMART POWER SUPPLY HOUSING AND DISPLAY

(71) Applicant: DePuy Synthes Products, Inc., Raynham, MA (US)

(72) Inventors: Adrian BAUMGARTNER, Raynham, MA (US); Nicholas BOWERS, Raynham, MA (US); John CONIDI, Raynham, MA (US); Garrett GOODE, Raynham, MA (US); Jacob A. MARKS, Raynham, MA (US); David NARDUCCI, Raynham, MA (US); Elizabeth SCHLUETER, Raynham, MA (US); Jae S. STELZER, Raynham, MA (US); Thomas HAENGGI, Münchenstein (CH); Matthias SINGEISEN, Itingen (CH); Martin REINBERG, Bubendorf (CH); Dominic HÄCHLER, Windisch (CH); Dirk HOYER, Hölstein (CH); Bruno ALESSANDRI, Sempach (CH); Patrick UHRI, Reigoldswil (CH); Markus HERMANN, Laupersdorf (CH)

(21) Appl. No.: 19/315,046

(22) Filed: Aug. 29, 2025

Related U.S. Application Data
(60) Provisional application No. 63/701,000, filed on Sep. 30, 2024.
(60) Provisional application No. 63/821,159, filed on Jun. 10, 2025.

(51) Int. Cl. B25F 5/02; G06F 3/04842

(52) U.S. Cl. CPC B25F 5/02; G06F 3/04842

(57) ABSTRACT

A smart hand-held power tool system including a handpiece with a tool head performing an operation and a smart power supply housing connectable to the handpiece controlling at least one parameter setting and/or functionality associated with the operation of the smart hand-held power tool system. The smart power supply housing includes a power supply enclosed therein; and a screen displaying the at least one parameter setting and/or the functionality associated with the operation of the smart hand-held power tool system. Functionality performed by the smart power supply housing may include advanced assistant features such as navigation, geofencing and/or anti-kickback based on navigation, acceleration and/or torque measurements generated by an Inertial Measurement Unit associated with the smart power supply housing based on feedback data detected by sensor(s).

### CROSS REFERENCE TO RELATED APPLICATIONS

This application claims the benefit of priority under 35 U.S.C. § 119 to prior filed U.S. Provisional Ser. No. 63/701,000 , filed Sep. 30, 2024 (Attorney Docket No.: 267214.000002 (DSP6433USPSP1)) and to prior filed U.S. Provisional Ser. No. 63/821,159 , filed Jun. 10, 2025 (Attorney Docket No.: 267214.000006 (DSP6433USPSP2)), the entire contents of both applications of which are hereby incorporated by reference in their entirety as if set forth in full herein.

### FIELD

The present disclosure generally relates to a smart (i.e., intelligent) power tool system such as that used for performing orthopedic operations (e.g., drilling, sawing, oscillating sawing, reciprocating sawing, impacting, etc.). In particular, the present disclosure is directed to a smart (i.e., intelligent) power tool system including a handpiece and a smart (i.e., intelligent) power supply housing for controlling and displaying parameter settings and/or functionality associated with operation of the smart power tool system. Such functionality may include advanced/enhanced assistance feature (e.g., navigation, geofencing, anti-kickback, current insertion depth based on navigation, acceleration and/or torque measurements generated by an Inertial Measurement Unit based on feedback data detected by sensor(s)).

### BACKGROUND

In the field of orthopedics, hand-held power tools performing a variety of operations (e.g., drilling, sawing, impacting, or other functions performed by a power tool) are often used during surgical joint replacement procedures (e.g., knee, shoulder or hip replacement, arthroscopy, spine procedures, craniomaxialfacial procedures, etc.). Powered orthopedic tools provide high accuracy and efficiency in comparison to manual orthopedic tools.

With conventional hand-held power tools, the only functionality associated with the power supply housing is powering the handpiece. In addition, in current orthopedic power tool systems enhanced/advanced assistant features such as navigation, torque and acceleration measurements when provided are performed via auxiliary components, modules or devices other than the handpiece or the power supply housing.

It is desirable to develop an improved smart (i.e., intelligent) hand-held power tool system including a handpiece and a smart (i.e., intelligent) power supply housing (e.g., battery housing or battery pack) controlling parameter settings and/or functionality while also providing enhanced/advanced assistant features such as navigation, acceleration and/or torque measurements based on feedback data detected by sensors making the system more user-friendly while minimizing potential safety risks to both the medical professional and the patient.

### SUMMARY

An aspect of the present disclosure is directed to an improved smart hand-held power tool system. The system includes a handpiece, a smart power supply housing, and a display. The handpiece includes a tool head for performing an operation and at least one sensor. The smart power supply housing is connectable to the handpiece and controls at least one parameter setting or functionality associated with the operation of the smart hand-held power tool system. The smart power supply housing includes a power supply enclosed within the smart power supply housing and housing circuitry. The housing circuitry (i) produces navigation, speed and/or torque outputs based on feedback data generated by the at least one sensor associated with the handpiece, the smart power supply housing and/or the tool head (100), and (ii) controls at least one parameter setting associated with the operation of the handpiece. The display is in communication with the smart power supply housing and includes a graphical user interface display circuitry. The display circuitry is configured to display a plurality of interfaces on the graphical user interface. At least one interface of the plurality of pages includes the at least one parameter setting.

An aspect of the present disclosure is directed to a method of controlling handpieces for performing one or more operations. The method includes receiving a selection, to an external display from a first user of a first handpiece from a connection menu, the first handpiece being available on the connection menu in response to connection of a smart power supply housing to the first handpiece, and the selection comprising a request to wirelessly connect the external display to the smart power supply housing. The method includes, based on the selection of the first handpiece from the connection menu, transmitting a prompt to the smart power supply housing to confirm the request to wirelessly connect the external display to the smart power supply housing. The method includes receiving a confirmation, to the smart power supply housing from a second user, to wirelessly connect the external display to the smart power supply housing. The method includes wirelessly connecting the external display to the smart power supply housing to permit information to be transmitted between the external display to the smart power supply housing, the information comprising one or smart features supported by the first handpiece.

### BRIEF DESCRIPTION OF THE DRAWINGS

The above and further aspects of the present disclosure are further discussed with reference to the following description in conjunction with the accompanying drawings, in which like numerals indicate like structural elements and features in various figures. The drawings are not necessarily to scale, emphasis instead being placed upon illustrating principles of the present disclosure. The figures depict one or more implementations of the devices, by way of example only, not by way of limitation.

FIG. 1A is a side view of the smart (i.e., intelligent) orthopedic hand-held power system in accordance with the present disclosure including only the handpiece (with the interchangeable tool head releasably attachable thereto) and the smart (i.e., intelligent) power supply housing depicted assembled/connected/installed together;

FIG. 1B is a rear-side perspective view of the smart (i.e., intelligent) orthopedic hand-held power tool system of FIG. 1A depicting the graphical user interface displayed on the screen/monitor integrated into the rear sidewall (opposite that of the tool head) of the smart (i.e., intelligent) power supply housing;

FIG. 1C depicts a rear-downward-side perspective view of the power supply housing alone of the smart (i.e., intelligent) orthopedic hand-held power system of FIG. 1A depicting integrated into the rear sidewall (opposite that of the tool head) of the smart (i.e., intelligent) power supply housing both the graphical user interface displayed on the screen/monitor and physically manipulatable control buttons;

FIG. 1D depicts a partial rear-upward-side perspective view of the smart (i.e., intelligent) power supply housing and lower portion of the handpiece releasably connected thereto via the releasable latch of the smart (i.e., intelligent) orthopedic hand-held power tool system of FIG. 1A illustrating integrated into the rear sidewall (opposite that of the tool head) of the smart (i.e., intelligent) power supply housing both the graphical user interface displayed on the screen/monitor and physically manipulatable control buttons;

FIG. 2 is an exemplary schematic electronic circuitry diagram of the smart (i.e., intelligent) power supply housing and an external display of the smart (i.e., intelligent) orthopedic hand-held power supply tool system in accordance with the present disclosure;

FIG. 3 is a first exemplary graphical user interface of the external display in accordance with the present disclosure;

FIG. 4 is a second exemplary graphical user interface of the external display in accordance with the present disclosure;

FIG. 5 is a third exemplary graphical user interface of the external display in accordance with the present disclosure;

FIG. 6A is a fourth exemplary graphical user interface of the external display in accordance with the present disclosure;

FIG. 6B is a secondary screen of the fourth exemplary graphical user interface of the external display in accordance with the present disclosure;

FIGS. 7A-7D are a fifth exemplary graphical user interface of the external display in accordance with the present disclosure;

FIG. 8A is a view of a drill bit drilled into a bone to a depth;

FIG. 8B is a sixth exemplary graphical user interface of the external display in accordance with the present disclosure;

FIG. 9A is a view of a drill bit reaching the second cortex of a bone;

FIG. 9B is a seventh exemplary graphical user interface of the external display in accordance with the present disclosure;

FIG. 10 is a rear view of an alternative power supply housing a lower portion of the handpiece in accordance with the present disclosure;

FIG. 11A is a perspective view of another exemplary smart (i.e., intelligent) orthopedic hand-held power system in accordance with the present disclosure;

FIG. 11B is a detail view of the exemplary smart (i.e., intelligent) orthopedic hand-held power system of FIG. 11A in accordance with the present disclosure, depicting a rotatable hinged display;

FIG. 11C is a detail view of the exemplary smart (i.e., intelligent) orthopedic hand-held power system of FIG. 11A in accordance with the present disclosure, depicting the detachability of the rotatable hinged display;

FIG. 12A is a perspective view of another exemplary smart (i.e., intelligent) orthopedic hand-held power system in accordance with the present disclosure;

FIG. 12B is a side view of the exemplary smart (i.e., intelligent) orthopedic hand-held power system of FIG. 12A in accordance with the present disclosure, depicting a hinged display tethered to a smart power supply housing;

FIG. 12C is a perspective view of the hinged display and smart power supply of FIG. 12A in accordance with the present disclosure;

FIG. 13A is a perspective view of another exemplary smart (i.e., intelligent) orthopedic hand-held power system in accordance with the present disclosure;

FIG. 13B is a perspective view of the hinged display and smart power supply of FIG. 12A in accordance with the present disclosure, showing the display inserted into a recess in the battery;

FIG. 14A is a perspective view of a docking station in accordance with the present disclosure;

FIG. 14B is a detail view of various exemplary digital dashboards of the docking station of FIG. 14A in accordance with the present disclosure;

FIG. 15 is a perspective view of a smart (i.e., intelligent) orthopedic hand-held power tool system that includes audible feedback in accordance with the present disclosure;

FIG. 16 is a perspective view of a smart (i.e., intelligent) orthopedic hand-held power tool system that includes tactile feedback in accordance with the present disclosure;

FIGS. 17A-17C are detail views of a smart (i.e., intelligent) orthopedic hand-held power tool system that include light indicators in accordance with the present disclosure;

FIG. 18A is a perspective view a smart (i.e., intelligent) orthopedic hand-held power tool system that includes a removable light indicator in accordance with the present disclosure;

FIG. 18B is a perspective view of the smart (i.e., intelligent) orthopedic hand-held power tool system of FIG. 18A, with the light indicator removed and docked with a smart power supply housing;

FIG. 19A is a detail view of the handpiece of FIG. 1A, shown with portions thereof cutaway to illustrate a speed selector in a first mode and a speed selector sensor in accordance with the present disclosure;

FIG. 19B is a detail view similar to FIG. 19, illustrating the speed selector in a second mode in accordance with the present disclosure;

FIG. 20 is a cross-sectional view of FIG. 19B, cut relative to a vertical plan that intersects an axis of a plunger rod, in accordance with the present disclosure;

FIG. 21 is a cross-sectional view, similar to that of FIG. 20, of the handpiece of FIG. 1A with an alternative speed selector and speed selector sensor in accordance with the present disclosure; and

FIG. 22 is an exemplary sequence diagram for wireless communication between the smart power supply housing and the external display in accordance with the present disclosure.

### DETAILED DESCRIPTION

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±20% of the recited value, e.g. “about 90%”may refer to the range of values from 71% to 99%.

As used herein, the terms “component,” “module,” “system,” “server,” “processor,” “memory,” and the like are intended to include one or more computer-related units, such as but not limited to hardware, firmware, a combination of hardware and software, software, or software in execution. For example, a component may be, but is not limited to being, a process running on a processor, an object, an executable, a thread of execution, a program, and/or a computer. By way of illustration, both an application running on a computing device and the computing device can be a component. One or more components can reside within a process and/or thread of execution and a component may be localized on one computer and/or distributed between two or more computers. In addition, these components can execute from various computer readable media having various data structures stored thereon. The components may communicate by way of local and/or remote processes such as in accordance with a signal having one or more data packets, such as data from one component interacting with another component in a local system, distributed system, and/or across a network such as the Internet with other systems by way of the signal. Computer readable medium can be non-transitory. Non-transitory computer-readable media include, but are not limited to, random access memory (RAM), read-only memory (ROM), electronically erasable programmable ROM (EEPROM), flash memory or other memory technology, compact disc ROM (CD-ROM), digital versatile disks (DVD) or other optical storage, magnetic cassettes, magnetic tape, magnetic disk storage or other magnetic storage devices, or any other tangible, physical medium which can be used to store computer readable instructions and/or data.

As used herein, the term “computing system” is intended to include stand-alone machines or devices and/or a combination of machines, components, modules, systems, servers, processors, memory, detectors, user interfaces, computing device interfaces, network interfaces, hardware elements, software elements, firmware elements, and other computer-related units. By way of example, but not limitation, a computing system can include one or more of a general-purpose computer, a special-purpose computer, a processor, a portable electronic device, a portable electronic medical instrument, a stationary or semi-stationary electronic medical instrument, or other electronic data processing apparatus.

As used herein, the term “non-transitory computer-readable media” includes, but is not limited to, random access memory (RAM), read-only memory (ROM), electronically erasable programmable ROM (EEPROM), flash memory or other memory technology, compact disc ROM (CD-ROM), digital versatile disks (DVD) or other optical storage, magnetic cassettes, magnetic tape, magnetic disk storage or other magnetic storage devices, or any other tangible, physical medium which can be used to store computer readable information.

Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

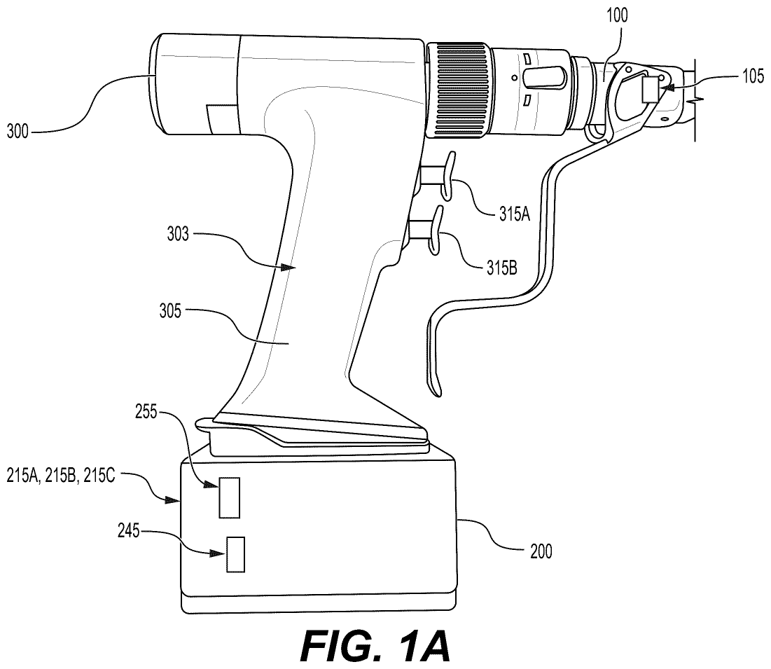

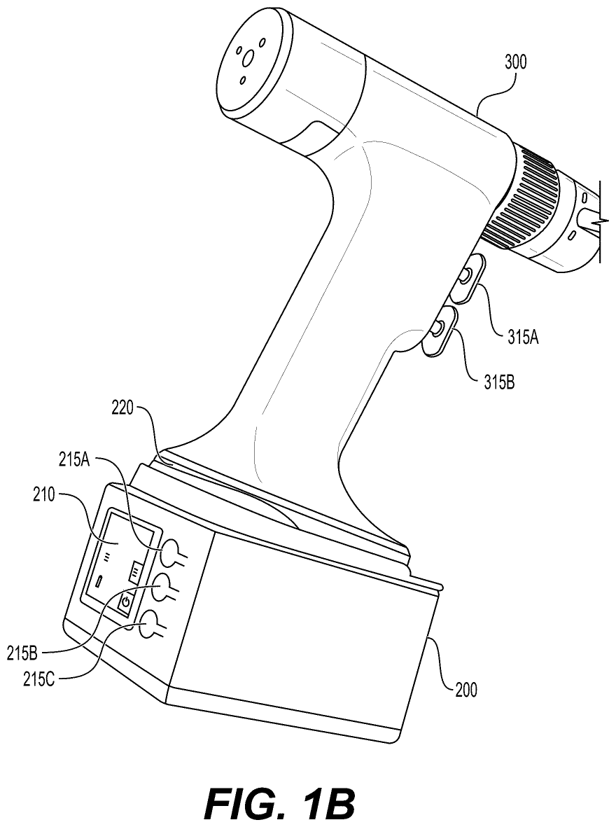

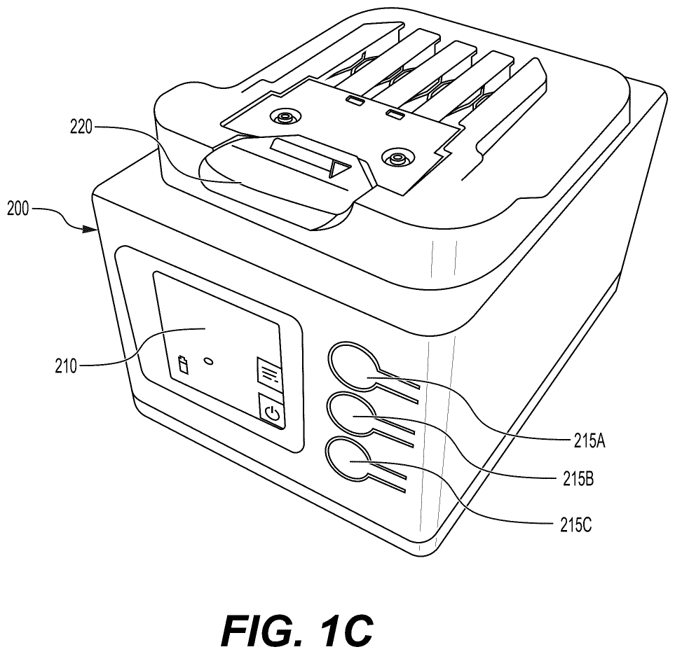

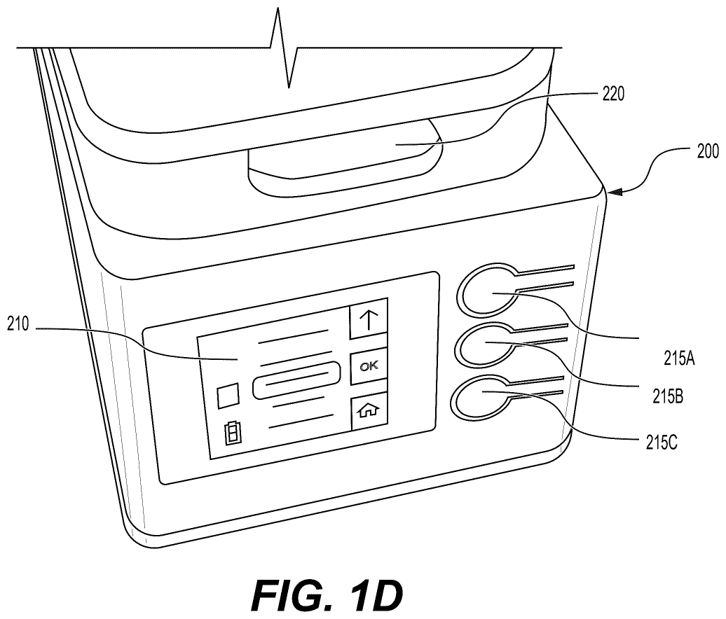

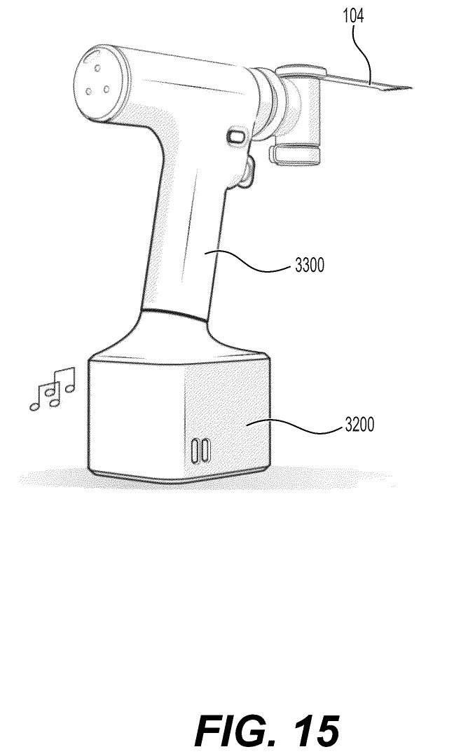

The present disclosure is directed to a smart (i.e., intelligent) hand-held power tool system such as that used during orthopedic surgical replacement procedures, typically hip or knee replacement. For purposes of illustration and description of the present disclosure the operation performed by the hand-held power tool system is an orthopedic operation such as an oscillating saw (e.g., FIG. 15), however, any desired power tool operation is contemplated and within the scope of the present invention. FIG. 1A is a side view of the smart (i.e., intelligent) orthopedic hand-held power tool system in an assembled state (i.e., connected, attached or installed) including a smart (i.e., intelligent) power supply housing 200 releasably attached to a handpiece 300 with a tool head 100 (preferably interchangeable) attached thereto (e.g., via release latch 220). The smart (i.e., intelligent) orthopedic hand-held power tool system in accordance with the present disclosure is simplified or streamlined eliminating the need for auxiliary additional smart (i.e., intelligent) auxiliary components or modules associated with processing advanced/enhanced assistant functionality (e.g., navigation, torque, and acceleration measurement based on feedback data detected by sensors). When installed, assembled or connected, complementary electrical contact terminals associated with each of the handpiece 300 and the smart (i.e., intelligent) power supply housing 200 are engaged and electrically connected with one another.

Addressing each of the components separately, the tool head 100 performs one or more orthopedic operations, e.g., drilling, reaming, oscillation drilling, sagittal sawing, reciprocating sawing or impacting. By way of illustrative example, the tool head shown is a pin driver attachment, but any desired orthopedic operation is possible. One or more sensors 105, preferably more than one, are arranged on the tool head 100, the handpiece 300 and/or the power supply housing 200. Sensors 105 monitor or detect feedback data that is received and processed by the smart (i.e., intelligent) power supply housing 200. By way of illustrative examples, sensor(s) 105 may be: (i) accelerometer(s) monitoring linear positioning; (ii) gyroscope(s) monitoring rotational motion; (iii) optical imaging sensor(s) (e.g., reflective glass bodies or Light Emitting Diodes (LEDs)) monitoring spatial positioning when picked up by an external image processing device (e.g., camera); (iv) electromagnet(s); and/or (v) magnetometer(s) monitoring magnetic field. A sensor 105 may be associated with each of three coordinate axes perpendicular to one another (e.g., x-axis, y-axis, z-axis). Acceleration sensors (e.g., accelerometers or gyroscopes) may be placed in different locations on the tool head 100, the handpiece 300 and/or the power supply housing 200. Preferably, the acceleration sensor is positioned as close as possible to the tool head 100 (e.g., on the tool head 100 or on the handpiece 300 proximate the tool head 100). An additional acceleration sensor, preferably located furthest away from the axis of the motor 303 (e.g., in the handpiece 300 or otherwise in the power supply housing 200), may be employed to detect twisting around the motor axis during reaming due to a sharp increase in torque stopping operation of the tool as a safety feature. The optical image sensors may be employed with an external image processing navigation system including an external camera(s). Feedback data detected by the optical image sensors is processed by the algorithm associated with the external image processing navigation system to determine the spatial position of the power tool displayable on a screen/monitor (e.g., associated with the smart power supply housing and/or external supplemental screen/monitor), and/or, when appropriate, stopping operation of the tool as a safety feature. Different tool heads 100, each performing a unique orthopedic operation, may preferably be interchangeably fitted on to the handpiece 300 via any conventional releasable securement mechanism (e.g., radially constricting collar).

Handpiece 300 has at least one trigger for controlling operating speed of the tool head 100 depending on the extent of squeezing (i.e., depressing) of the trigger by the user. In the example of FIGS. 1A-1D handpiece 300 has two triggers 315a, 315b independently operable of one another. Operating speed of the tool head 100 is controlled in the forward direction by one trigger and in the reverse direction via the other trigger. Alternatively, operating speed may be controlled via a single trigger with the direction (e.g., forward or reverse) selected by a separate toggle switch or the like.

Parameter settings and/or functionality associated with operation of the smart hand-held power tool system may be controlled by the smart (i.e., intelligent) power supply housing 200 (i.e., battery housing or battery pack) enclosing therein a power supply 205 (e.g., battery, preferably rechargeable). When the smart orthopedic hand-held power tool system is assembled, the power supply 205 (e.g., battery) provides the energy to power a motor 303 associated with the handpiece 300 that, in turn, operates the tool head 100. The smart (i.e., intelligent) power supply housing 200 includes software for controlling/processing intelligent functionality of the system (e.g., controlling hardware features, displaying outcomes, controlling the handpiece for geofencing, navigation, torque/speed limiting, controlling maximum speed/torque setting, enabling/disabling anti-kickback functionality, depth measuring, controlling maximum depth setting processing). Preferably, the smart power supply housing 200 also includes software for processing advanced/enhanced assistant features such as navigation, acceleration and/or torque measurements based on feedback data generated by the sensor(s) 105, thereby eliminating the need for auxiliary components/modules to perform such functions. Preferably, the smart power supply housing 200 features an integrated screen or monitor 210 on the external surface of its sidewall, which displays a graphical user interface Interaction with the graphical user interface may be realized either via the screen/monitor itself (e.g., as a touch screen) and/or via one or more physically manipulatable buttons 215a, 215b, 215c separate from the screen/monitor 210 associated with the smart (i.e., intelligent) power supply housing 200. Preferably, the physically manipulatable buttons are also integrated into the same external surface of the sidewall of the smart power supply housing 200 as that of the screen/monitor 210. In the example of FIGS. 1A-1D, the smart power supply housing 200 has three physically manipulatable buttons 215a, 215b, 215c. Any number of physically manipulable buttons may be included the location, size, shape, arrangement, functionality, etc. of each may be configured, as desired. The three physically manipulatable buttons in the example depicted represent the following respective operations: (i) incrementally adjusting (e.g., increasing/decreasing) the value of a control parameter or advancing (e.g., forward or reverse) through a menu of available options/modes/features and/or (ii) selection of a particular item or option in the menu of available options/modes/features. By way of example the uppermost physical manipulatable button 215c is increasing/advancing forward; the middle physically manipulatable button 215b is for decreasing/advancing in reverse; while the lowermost physically manipulatable button 215a is for selection of a particular item/option from a menu displayed on the screen/monitor 210. What operation/function is controlled by each physically manipulatable button may also be selected, as desired. Handpiece 300 includes a motor controller 305 for controlling operation (e.g., speed and/or direction) of the motor 303 moving the tool head 100 based on the signals generated by the triggers 315a, 315b. Other electronic components/circuitry/modules may be included with the handpiece 300.

Displayed on the screen/monitor 210 is the graphical user interface that may be designed, as desired. By way of illustration only, the graphical user interface displayed on the screen/monitor 210 may include information relating to: (i) device settings, (ii) current and/or adjusted status of one or more parameters associated with the device; and/or (iii) menu of available options/modes/features. For instance, the operations and information displayed via the graphical user interface may include: (i) home; (ii) current power status (e.g., ON/OFF); (iii) menu of selectable options/modes/features; (iv) current operating parameter(s) (e.g., speed/torque or depth of penetration); (v) maximum operating parameter setting (e.g., maximum speed/torque or maximum depth of penetration); (vi) current battery charge status (i.e., remaining battery life); (vii) current wireless connection status (e.g., Bluetooth connected/disconnected); (viii) current geofencing/navigation data (e.g., location—either absolute or relative) (ix) enabling/disabling enhanced assistant features/processing/systems (e.g., anti-kickback, geofencing, navigation, etc.); (x) warning(s) (e.g., current depth of penetration exceeds maximum depth of penetration setting or current speed/torque exceeds maximum speed/torque setting) and/or (xi) error(s). Parameter settings and/or functionality displayed on the screen 205 and/or controlled via the smart power supply housing 200 may be configured, as desired.

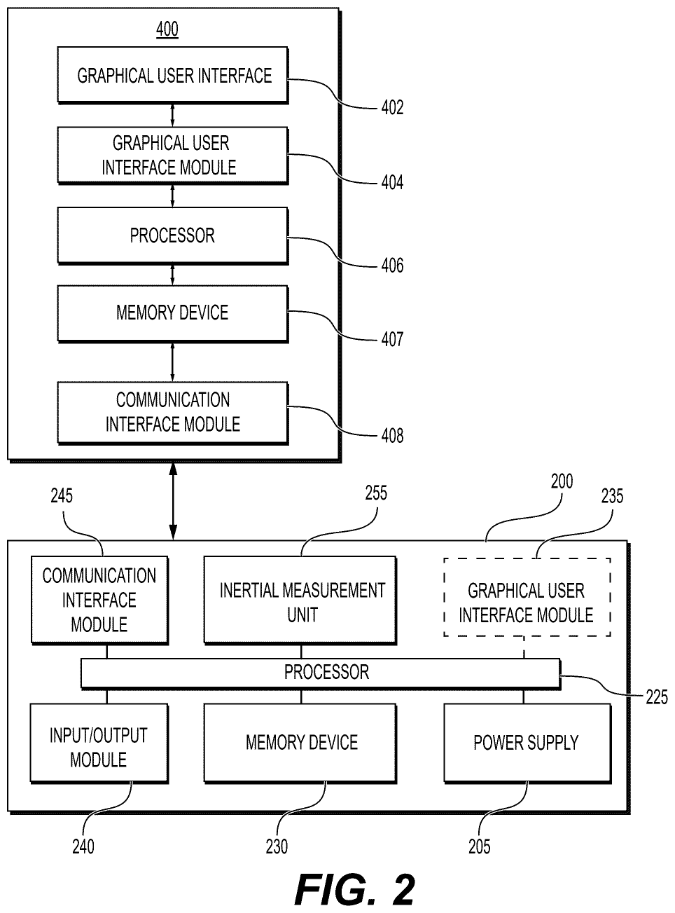

FIG. 2 is an example schematic electronic circuit diagram for the smart (i.e., intelligent) power supply housing 200 in which is disposed the power supply or power source 205 (e.g., battery, preferably rechargeable). Processor or controller 225 (e.g., CPU) produces control signals that control all other electronic components associated with the power supply housing 200. An associated memory or storage device 230 (e.g., ROM, RAM, EPROM) stores the applications and software for operating the processor or controller 225 and other electronic components/modules of the smart (i.e., intelligent) power supply housing 200. One or more electronic modules are in electronic communication with the processor 225. Power supply housing 200 may optionally include a wireless communication interface module 245 for wireless communication (e.g., Bluetooth or Wi-Fi). Graphical user interface module 235 displays and updates the graphical user interface on the screen/monitor 210. By way of example, the graphical user interface module 235 may update what is being displayed on the screen/monitor 210 based on: (i) user input via the touch screen and/or one or more manipulatable physical buttons 215a, 215b, 215c received by the input/output module 240; and/or (ii) navigation, acceleration, speed, and/or torque outputs produced/calculated by the Inertial Measurement Unit module 255 based on feedback data detected by the sensor(s) 105 associated therewith.

Feedback data detected, measured or monitored by the one or more navigation sensors 105 is processed by the Inertial Measurement Unit module 255 of the smart (i.e., intelligent) power supply housing 200 using motion fusion algorithms or software to produce navigation, acceleration, speed, and/or torque measurements. It is this navigation, acceleration, speed, and/or torque outputs generated/produced/calculated by the Inertial Measurement Unit module 255 of the power supply housing 200 that, in turn, may be used to update information displayed (e.g., current speed) on the screen/monitor 210. It is also contemplated to use the generated navigation, acceleration, speed and/or torque data for advanced/enhanced assistant functionality such as geofencing and/or navigation displayed on the screen/monitor 210. Any deviation from a target/desired reference (e.g., working axis) may be corrected by the user or automatically based on the navigation, acceleration, speed, and/or torque outputs generated by the Inertial Measurement Unit module 255 and/or, optionally when appropriate, to stop operation of the tool as a safety measure. Still further the feedback data as well as the generated navigation, acceleration, speed and/or torque outputs may be instrumental in minimizing or reducing potential risk of injury to the user and/or patient when operating the tool head. Generated navigation, acceleration and/or torque outputs/measurements may be employed to reduce potential risk of injury, for example, limiting maximum depth insertion of the tool head in the body or anti-kickback functionality halting operation altogether in response to detecting potentially harmful kickback. Accordingly, the smart (i.e., intelligent) power supply housing 200 of the smart (intelligent) power tool system in accordance with the present disclosure includes all the hardware and associated software with controlling and/or operating the smart (i.e., intelligent) power tool system including advanced/enhanced assistant features (e.g., navigation, acceleration, speed and/or torque outputs based on received feedback data from the navigation sensor(s) 105). By way of example, the smart (i.e., intelligent) power supply housing 200 may include hardware (e.g., Bluetooth communication interface; Wi-Fi communication interface; Inertial Measurement Unit; interface to tools/instruments with navigation sensors, display, buttons, etc.) and associated software (e.g., controlling hardware; processing feedback data generated by the sensor(s); displaying information/data on the screen/monitor; controlling operation of the handpiece; geofencing; navigation; limiting maximum torque/speed; adjusting maximum torque/speed; anti-kickback protection; and/or measuring depth of penetration of tool head 100).

During operation of the smart hand-held power tool system, the smart (i.e., intelligent) power supply housing 200 processes and displays current parameter settings, control parameter settings and/or functionality associated with operation of the hand-held power tool system. Functionality performed by the smart (i.e., intelligent) power supply housing preferably includes advanced/enhanced assistant features such as navigation, geofencing, anti-kickback based on navigation, acceleration, speed and/or torque outputs/measurements generated by an Inertial Measurement Unit 255 based on feedback data received from sensor(s) 105 associated therewith.

A screen or monitor 210 associated with the power supply housing 200 displays status, parameter settings, functionality, warnings, and/or errors, etc. In some examples, the screen or monitor 210 is integrated into an external surface of a sidewall of the power supply housing 200, as seen in FIGS. 1C-1D. Available functionality (e.g., modes or features) associated with the operation of the hand-held power tool system may also be displayed on the screen or monitor 210 of the smart power supply housing 200 using a graphical user interface selectable via touch screen or physically manipulatable buttons associated with the smart power supply housing 200.

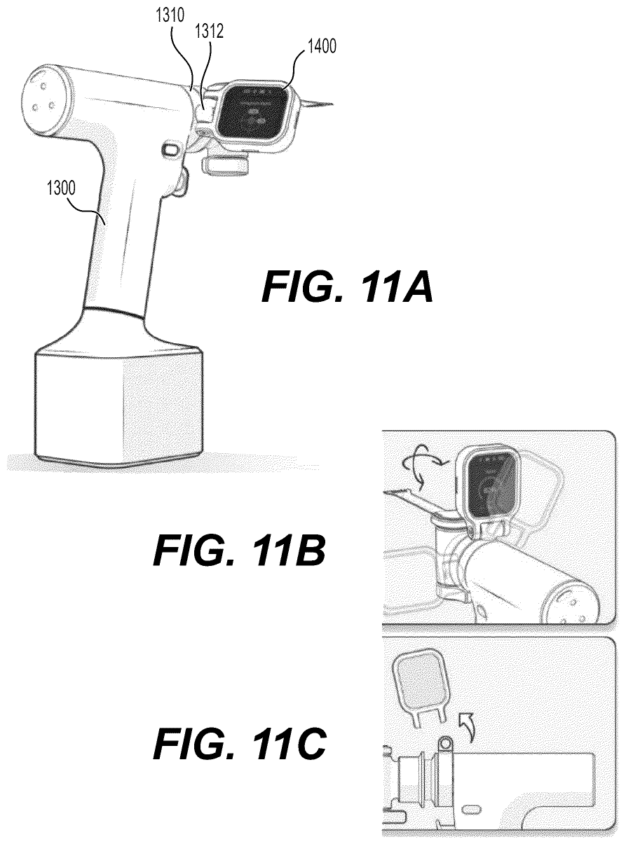

In other examples, as an alternative to or in addition to the screen or monitor 210, an external display 400 can be provided to display the aforementioned outputs, status, settings, functionality, warnings, errors, etc. The external display can be embodied as an off-board display detached from the handpiece 300. Doing so provides the capability for other users (besides the surgeon) to view and/or control features (with set parameters) of the handpiece 300 via two-way communication between the handpiece 300 and the external display 400. Other users include, but are not limited to, surgical technicians, nurses, sales representatives, and service or repair technicians. In alternative examples, as discussed in FIGS. 11A-13B, the external display can be embodied as an on-board display that is detachably and/or adjustably connected to the handpiece 300, providing improved visibility to the user of the handpiece 300 of the features enabled by smart power supply housing 200.

It is noted that the following software-enabled interfaces can be provided on a dedicated device (e.g., a portable computing device, a desktop computing device, a base station computing device, or a screen) or can be provided as an application installable on an end-user's computing device. The following examples of interfaces provide clear and simple feedback to the user(s) as well as intuitive ways to interact with all of the smart features of the presently described handpiece 300, including setting certain parameters of the handpiece 300 (which are discussed in greater detail below).

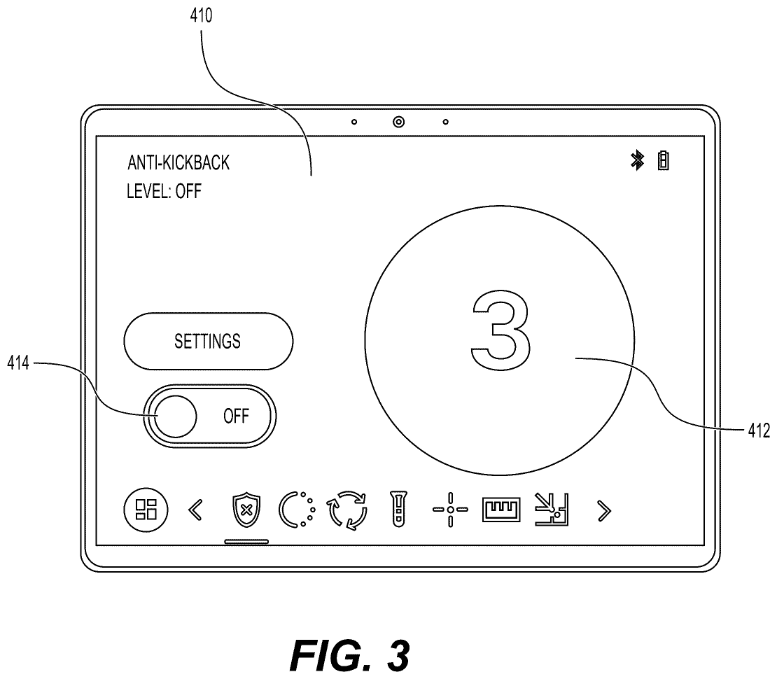

Returning to FIG. 2, in examples where an external display 400 is used with the handpiece 300, the external display 400 can include a graphical user interface 402, a graphical user interface module 404, a processor or controller 406, and a communication interface module 408. Processor or controller 406 (e.g., CPU) produces control signals that control all other electronic components associated with the external display 400. An associated memory or storage device 407 (e.g., ROM, RAM, EPROM) stores the applications and software for operating the processor or controller 406 and other electronic components/modules of the external display 400. One or more electronic modules are in electronic communication with the processor 406. The external display 400 includes a wireless communication interface module 410 for wireless communication (e.g., Bluetooth or Wi-Fi) with the wireless communication interface module 245 of the smart power supply housing 200. Graphical user interface module 404 displays and updates the graphical user interface 402 on the external display 400. FIGS. 3-7D, 8B, and 9B depict exemplary graphical user interfaces 402 in accordance with the disclosed technology, which are discussed in detail further herein below.

The processors and modules illustrated in FIG. 2 are implementable by circuitry including at least one semiconductor integrated circuit such as at least one processor (e.g., a central processing unit (CPU)), at least one application specific integrated circuit (ASIC), and/or at least one field programmable gate array (FPGA). At least one processor is configurable, by reading instructions from at least one machine readable non-transitory tangible medium, to perform at least some of the functions of the external display 400 and the smart power supply housing 200. Such a medium may take many forms, including, but not limited to, any type of magnetic medium such as a hard disk, any type of optical medium such as a CD and a DVD, any type of semiconductor memory (i.e., semiconductor circuit) such as a volatile memory and a non-volatile memory. The volatile memory may include a DRAM and a SRAM, and the nonvolatile memory may include a ROM and a NVRAM. The ASIC is an integrated circuit (IC) customized to perform, and the FPGA is an integrated circuit designed to be configured after manufacturing to perform, all or a part of the functions described herein.

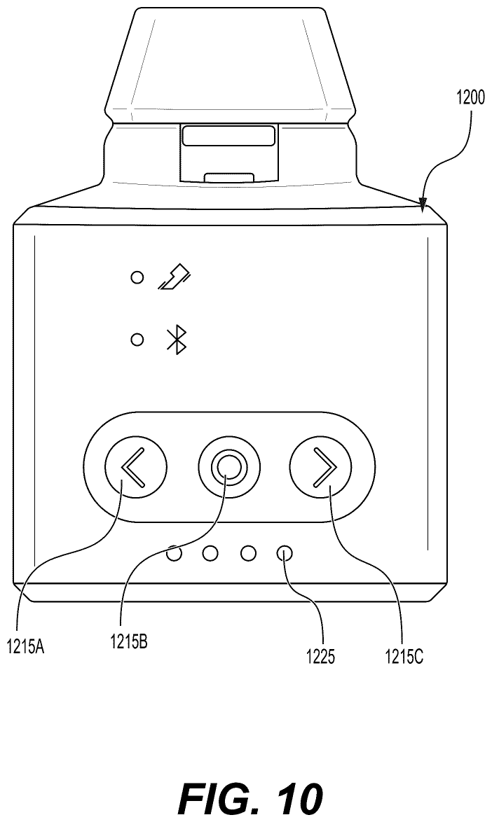

Turning now to FIG. 3, a first exemplary graphical user interface 410 is shown that is related to the aforementioned anti-kickback protection enabled by the handpiece 300, which reduces the risk of injury to the operator of the handpiece 300. Anti-kickback protection automatically stops the handpiece 300 (e.g., the motor is stopped or power to the tool head 100 is stopped), preventing kickback when a sudden unexpected movement occurs due to a stall event in, e.g., high torque reaming. This feature can be activated or deactivated via a toggle 414 on the first graphical user interface 410 or via a toggle/button 1215B (FIG. 10) on the smart power supply housing 200.

Moreover, a sensitivity level 412 of the kickback protection can be selected and adjusted by the user. These sensitivity levels 412 can each be associated with an angular acceleration value and/or velocity value of the handpiece 300 determined by a sensor (e.g., sensor 105). When a sensitivity level 412 is selected by a user of the external display 400, the communication interface module 408 of the external display 400 transmits the associated angular acceleration value or angular velocity value to the communication interface module 245 of the smart power supply housing 200 to set a threshold value to be monitored. Upon detection by the sensor 105 or the Inertial Measurement Unit 255 of the angular acceleration value or angular velocity value (e.g., the angular velocity value from the Inertial Measurement Unit 255) exceeding the threshold value (i.e., the associated angular acceleration value or angular velocity value of the selected sensitivity level), the processor 225 stops power delivery to the tool head 100 and/or stops the motor of the handpiece 300.

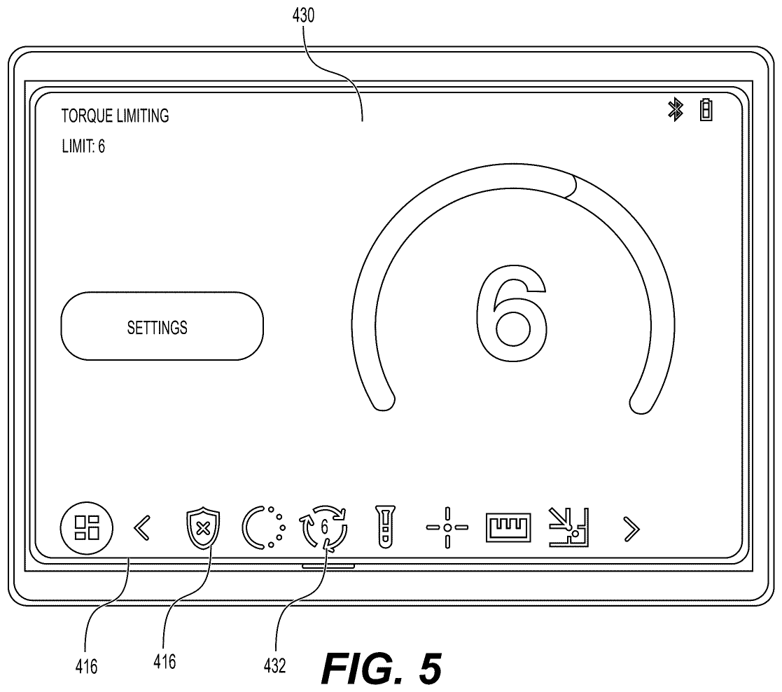

Various indicators can also be provided to the user(s) to provide them feedback regarding the status of this feature. For example, an icon or other indicator 416 (FIG. 5) can be presented on the graphical user interface 402 when the anti-kickback feature is activated. As seen best in FIG. 5, when active, a colored outline and icon highlight 416 alert to the user that the anti-kickback feature is activated in use. Alternatively, or in addition to, a light 1215 on the smart power supply housing 200 can be turned on by the processor 225 when the anti-kickback feature is activated.

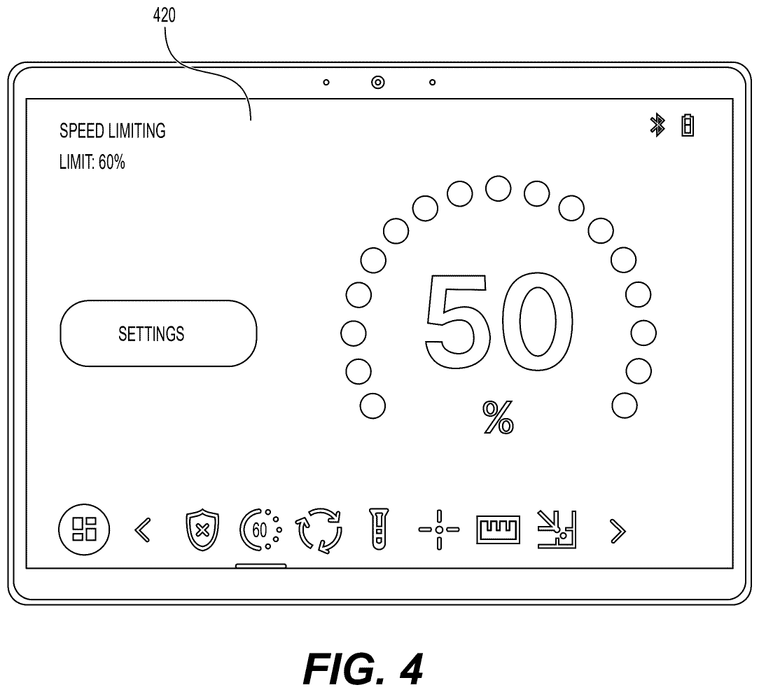

Turning now to FIG. 4, a second exemplary graphical user interface 420 is shown that is related to the aforementioned speed limiting feature that reduces the maximum speed of the tool head 100, improving surgical control. As seen in FIG. 4, a plurality of pre-determined levels can be presented to the user for selection. By way of example, the speed of the tool head 100 can be adjusted on the graphical user interface 420 from as low as 10% to as high as 100% in increments of 10%. Of course, more fine-tuned adjustment can be provided in accordance with the present disclosure.

Making reference to FIG. 5, a third exemplary graphical user interface 420 is shown that is related to the aforementioned torque limiting feature that limits the maximum torque applied the tool head 100, increasing surgical control. As seen in FIG. 5, a plurality of pre-determined levels can be presented to the user for selection. By way of example, the maximum output torque of the tool head 100 can be adjusted on the graphical user interface 420 from a minimum setting of 1 to a maximum setting of 10 in increments of 1 (such that there are 10 total maximum output torque settings), with a lighter line showing a maximum settable amount of the output torque. Of course, more fine-tuned adjustment can be provided in accordance with the present disclosure. Once the limit is selected, it can be displayed at the top of the graphical user interface 402. Once the limit is set, the torque limit icon 432 keeps the select value visible when the user navigates to different screens. Similar to the previously described anti-kickback feature, in some examples, a toggle switch can be provided to turn on and off the torque limiting feature.

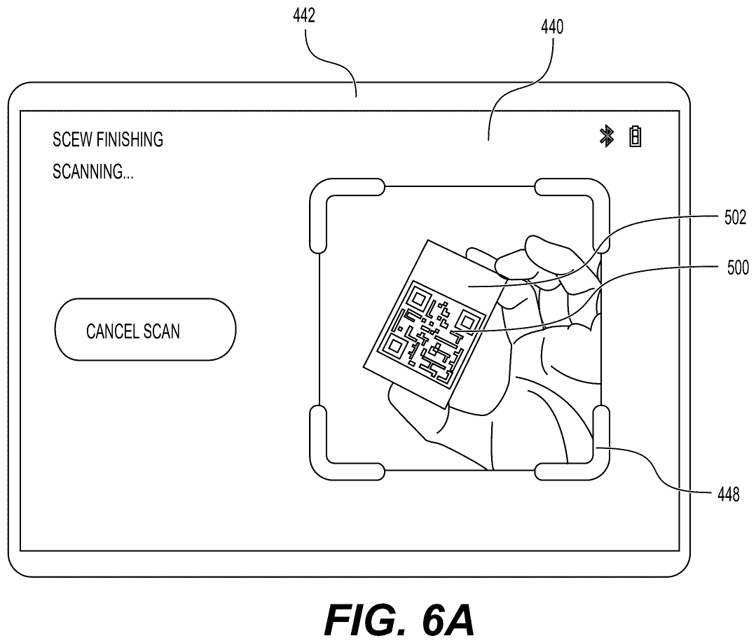

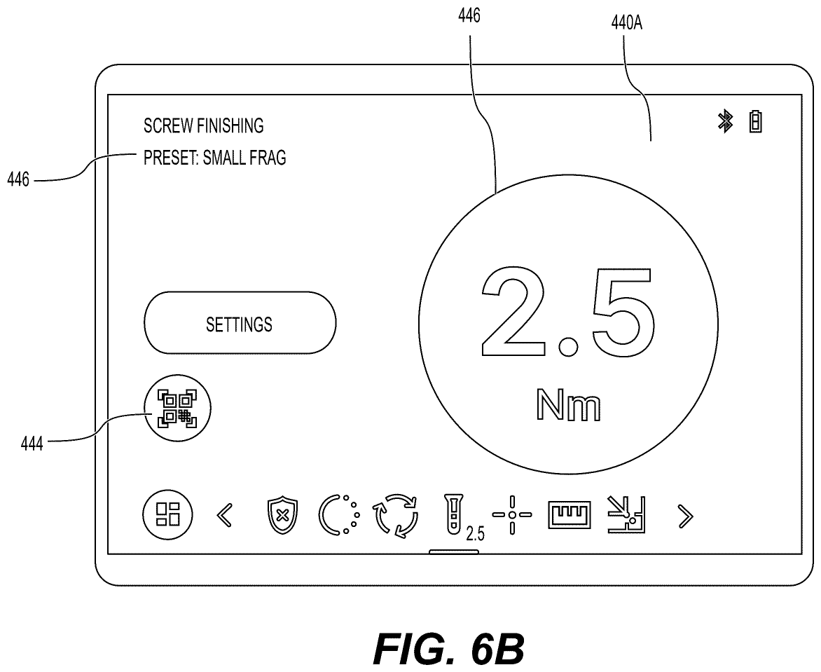

With reference now to FIGS. 6A-6B, a fourth exemplary graphical user interface 440 is shown that depicts an electronic screw finishing feature that provides for final tightening of screws by applying a prescribed torque. This feature can be used in place of manual torque limits and enables the handpiece 300 to automatically have the correct maximum torque output set on the tool head 100. This is achieved by the external display 400 being capable of identifying various screw types that are used during a procedure. More specifically, the external display 400 can include a sensor 442 for detecting a feature 500 associated with the screw. The feature 500 has identifying information about the screw type. In some examples, the sensor 442 includes a camera for scanning a Quick Response (QR) code (an example of a feature 500) on single use packaging 502 of the screw. As shown in FIG. 6A, the sensor 442 can be aligned with the QR code 500 using a region 448 (e.g., a box-shaped camera feed region) that visualizes the packaging 502 and aids in aligning the packaging 502 relative to the sensor 442.

With continued to reference to FIGS. 6A-6B, in order to identify the screw type, either memory device 407 or 230 can store a database of screw types (or QR codes associated with particular screw types) and torque output values of the tool head 100 associated therewith. Based on the identified screw type, the processor 225 of the smart power supply housing 200 sets the tool head 100 to the associated torque output value, within a predetermined tolerance.

Moreover, the processor 406 can include minimum time thresholds (e.g., at least one to two seconds) for detecting the QR code 500, to eliminate the risk of false scans of screw packaging 502 (e.g., packaging that may just happen to be in the view of the sensor 442 when it is activated). Put another way, the external display 400 can require detection of the feature 500 for a predetermined period of time before the feature 500 is registered.

Of course, those skilled in the art will appreciate that other techniques can be employed to identify the screw type without departing from the spirit and scope of the present disclosure. For example, the packaging 502 could include an antenna that communicates with an antenna in the external display 400 (like the functionality of near-field communication).

With reference to FIG. 6B, it is noted that the fourth graphical user interface 440 includes another screen 440A that has a selectable button 444 for activating the display sensor 442. The primary screen 440A also displays the screw type of the packaging 502 (once identified). In some examples, the primary screen 440 can also include a counter that tracks the number of screws used during the procedure. For example, every time a packaging is scanned during a procedure/operation, the screw total is updated to reflect the total number of screws scanned. In some further examples, the fourth graphical user interface 440 can include subsequent prompts to confirm whether further screws need to be scanned which can be based, e.g., on the type of operation. Using these techniques, the output torque of the handpiece 300 can be accurately and efficiently set.

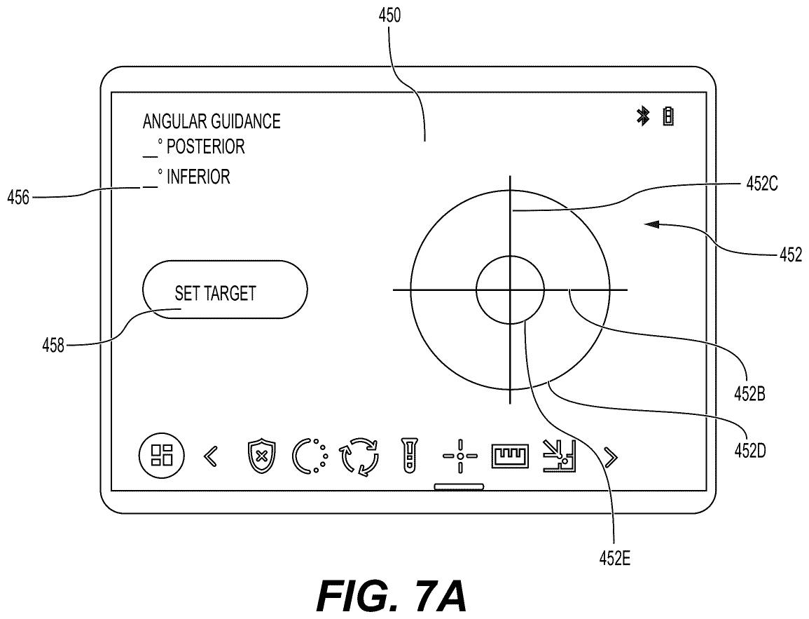

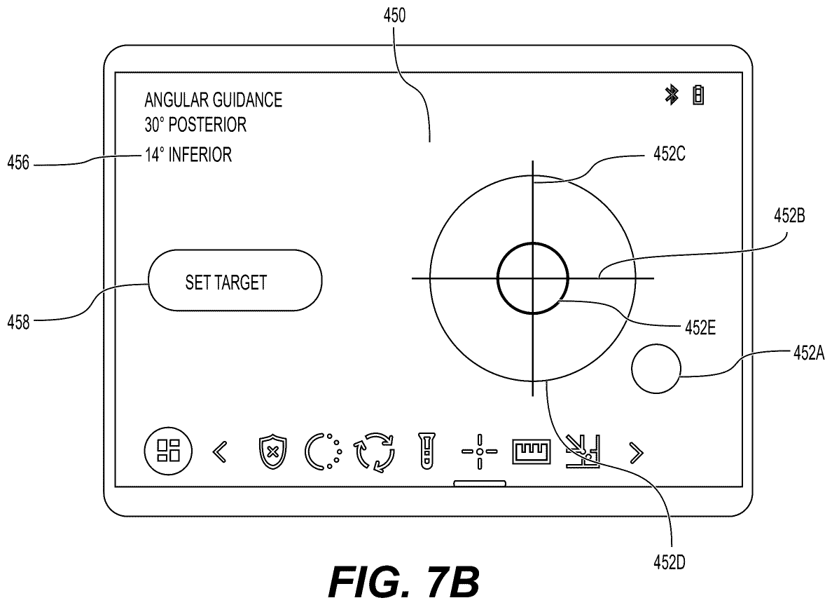

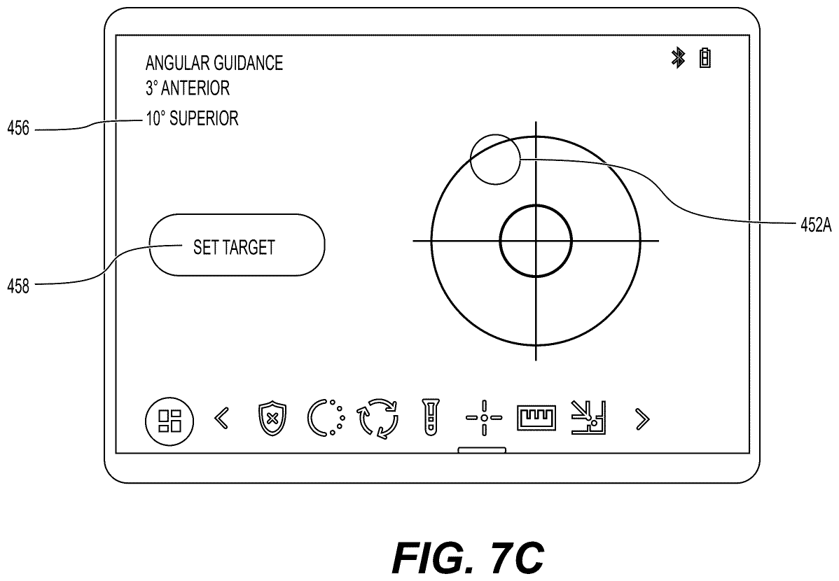

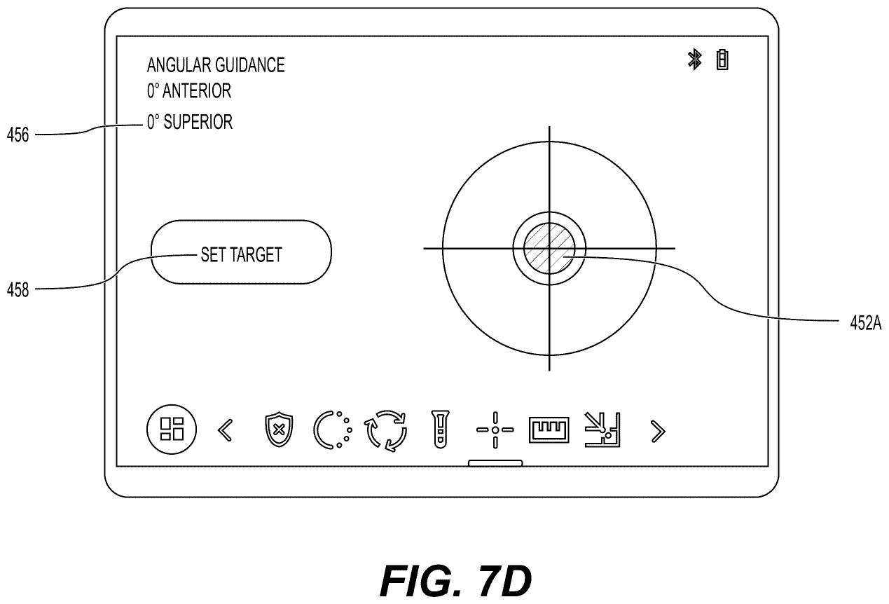

Turning now to FIGS. 7A-7D, a fifth exemplary graphical user interface 450 is shown that depicts an angular guidance feature that provides temporary trajectory guidance on a user-selected angle, increasing control and accuracy of the handpiece 300. For the angular guidance feature, all six values from the Inertial Measurement Unit are used (i.e., the 3D accelerometer and the 3D gyroscope). Sensor fusion is used to calculate the orientation trajectory of the handpiece 300 from these six values. The calculated orientation trajectory can then be transmitted to the external display 400.

This graphical user interface 450 includes a real-time guidance plot 452 with an indicator 452A representative of an orientation trajectory 456 of the tool head 100 relative to a target trajectory 454. The fifth graphical user interface 450 includes a button 458 for setting the target trajectory 454. Alternatively, a button 1215B (FIG. 10) on the smart power supply housing 200 can be pressed by the user of the handpiece 300 to set the target trajectory 454. Both the orientation trajectory 456 and the target trajectory 454 include a first angular component (e.g., an anterior component) and a second angular component (e.g., a superior component). The real-time guidance plot 452 includes a first target zone 452D and a second target zone 452E encompassed by the first target zone, with both target zones being indicative of varying levels of alignment of the orientation trajectory 456 relative to the target trajectory 454. While two target zones are employed in the present example, those skilled in the art will appreciate that any number of target zones can be employed without departing from the spirit and scope of the present disclosure. The guidance plot 452 can also be optionally divided into quadrants by lines 452B and 452C to provide enhanced visual feedback to the user of the external display 400 regarding the position of the handpiece 300.

Once the target trajectory 454 is set, the indicator 452A moves on the fifth graphical user interface 450 to provide real-time feedback to the user of the external display 400 regarding the positioning of the tool head 100 relative to the target trajectory 454, with the orientation trajectory 456 also updating in real-time.

FIG. 7B depicts a point in time after a user has selected a target trajectory 454. As seen, the indicator 452A falls outside the first target zone 452D, indicating the orientation trajectory 456 significantly deviates from the target trajectory 454. In this orientation, the indicator 452A can have a first appearance. Solely by way of example, the indicator 452A can have a light grey appearance when the indicator 452A is outside the first target zone 452D. In some examples, when the orientation trajectory 456 deviates from the target trajectory 454 more than a predetermined amount, the handpiece 300 or external display 400 can output an alert (e.g., an audible sound or a flashing light), which alert(s) the users that the handpiece 300 is significantly off its target trajectory 454.

FIG. 7C depicts another point in time after a user has selected a target trajectory 454. As seen, the indicator 452A falls outside the second target zone 452E but mostly within the first target zone 452D, indicating the orientation trajectory 456 deviates from the target trajectory 454, but is in closer alignment compared with the exemplary orientation of FIG. 7B. In this orientation, the indicator 452A can have a second appearance that differs from the aforementioned first appearance. Solely way of example, the indicator 452A can have a blue appearance when the indicator 452A is outside the second target zone 452E but a majority of the indicator 452A is within the first target zone 452D.

FIG. 7D depicts another point in time after a user has selected a target trajectory 454. As seen, the indicator 452A is aligned (or inside) the second target zone 452E, indicating the orientation trajectory 456 is aligned with the target trajectory 454 (within a certain tolerance). In this orientation, the indicator 452A can have a third appearance that differs from the aforementioned first and second appearances. Solely by way of example, the indicator 452A can be filled in blue, with the other lines of the guidance plot 452 also turning blue. In this way, the user(s) of the handpiece 300 and/or external display 400 can get instant feedback regarding the orientation of the handpiece 300.

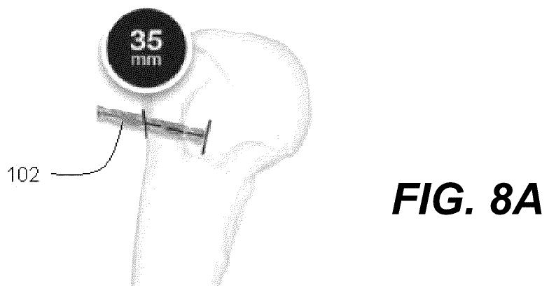

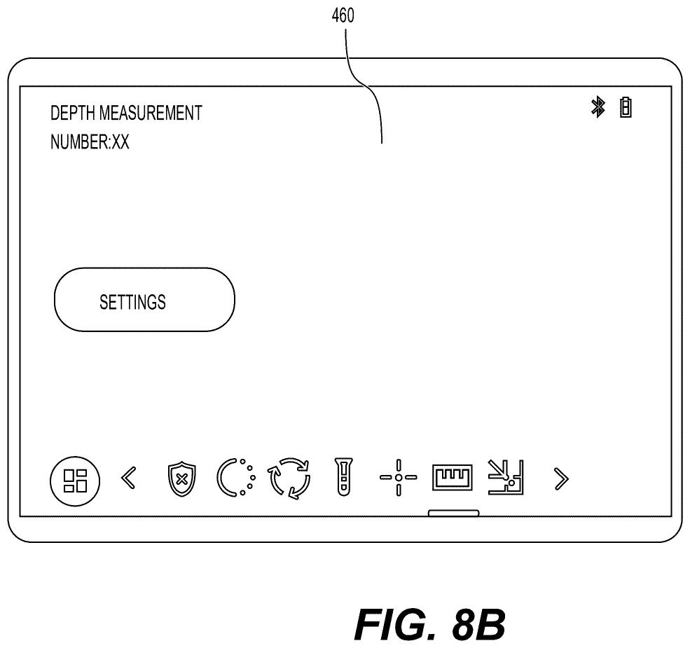

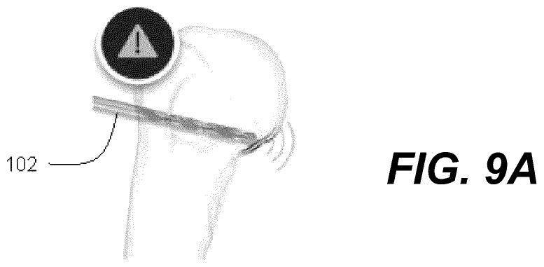

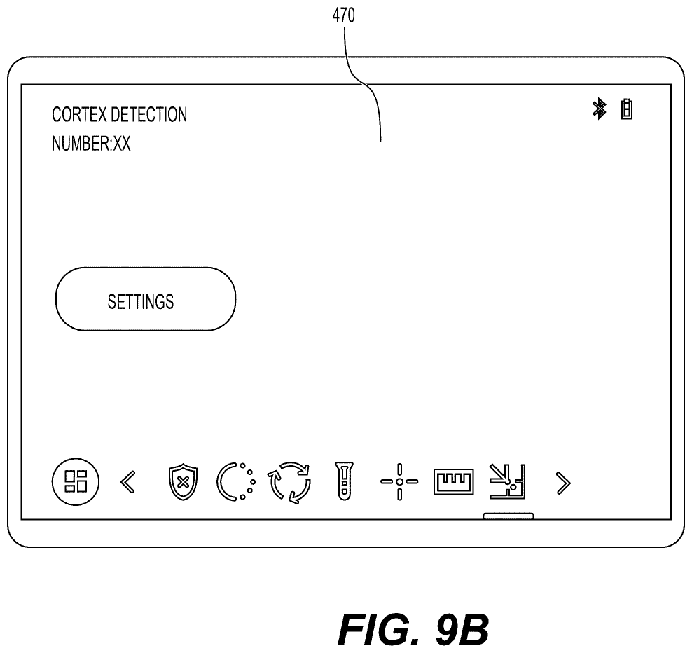

With reference to FIGS. 8A-8B, a sixth exemplary graphical user interface 460 (FIG. 8B) is shown that depicts a digital depth measurement feature that provides real-time readouts of the depth of drilling (exemplified in FIG. 8A) of a drill bit 102, which aids in screw selection. Similarly, with reference to FIGS. 9A-9B, a sixth exemplary graphical user interface 470 (FIG. 9B) is shown that depicts a second cortex detection feature that provides real-time feedback when the tip of the drill bit 102 reaches the second cortical wall (exemplified in FIG. 9A). This feature can be used to automatically select a bi-cortical screw based on the measured cortex to cortex length.

FIG. 10 illustrates an alternative power supply housing 1200 that functions equivalently to the previously described power supply housing 200. As seen, with the use of an external display 400, the power supply housing 1200 omits a screen but includes a plurality of buttons 1215A, 1215B, 1215C that can be used to toggle between selections/screens on the external display 400 (e.g., buttons 1215A and 1215C) and make selections on the display 400 (e.g., button 1215B). With this configuration, the user of the handpiece 300 can control the external display 400 without directly making contact with it, ensuring the external display remains sterile. Put another way, the power supply housing 1200 is capable of (i) receiving one or more inputs indicative of the selection of the at least one parameter setting (such as one of those previously mentioned, e.g., target trajectory, setting a speed or torque level, etc.) and (ii) transmitting the selection to the external display 400 for displaying the at least one parameter setting on the graphical user interface (e.g., the display 400 shows the target trajectory once set, it shows the torque or speed limit once set, etc.). Lights 1225 on the power supply housing 1200 can be used to show battery charge status, error in real-time, or various previously described alerts, etc.

Turning to FIGS. 11A-11C, and as discussed above, the external display can also be configured as an on-board display 1400 that is mountable to the handpiece 300 and one or both of removable or adjustable relative thereto. In this example, a ring 1310 is provided that is rotatably mounted on the handpiece 1300 (which is equivalent to the previously described handpiece 300), the on-board display 1400 is removably connected to the ring 1310 (see FIG. 11C). Removal of the on-board display 1400 enables it to be sterilized, as needed. A barrel 1312 can also be provided that enables the on-board display 1400 to pivot such that it has three degrees of freedom and the view angle can be easily adjusted.

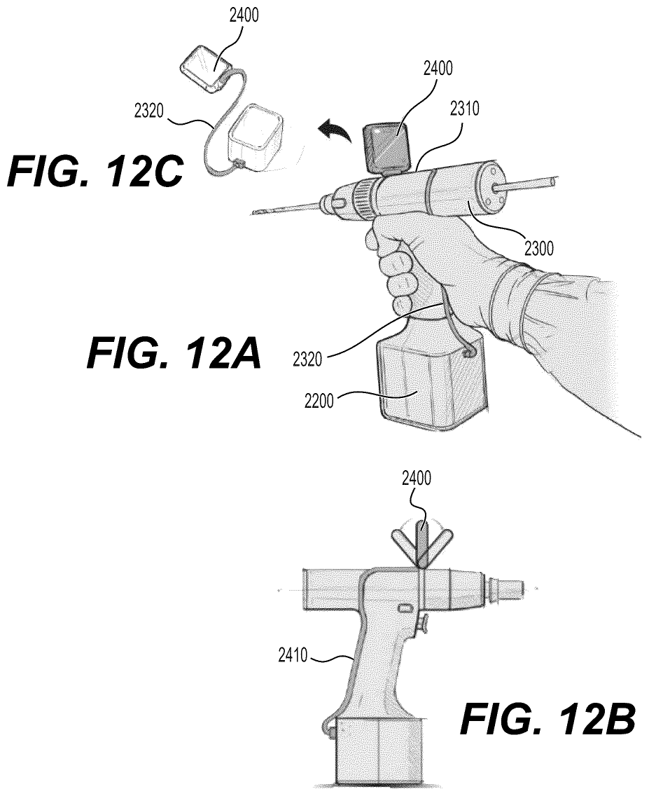

FIGS. 12A-12C depict another configuration of on-board external display 2400 that is connected to a handpiece 2300 (which is equivalent to the previously described handpiece 300) via a hinge 2310. In this example, the on-board display 2400 is tethered to the smart power supply housing 2200 (which is equivalent to the previously described smart power supply housing 200) via a cable 2410 that is routed along the handpiece 2300. The cable 2410 provides power to the on-board display 2400 from the smart power supply housing 2200 and, in some examples, facilitates data exchange therebetween. In some examples, the cable 2410 is routed along a groove 2320 in the handpiece 2300, which can be internal or external to the body of the handpiece 2300.

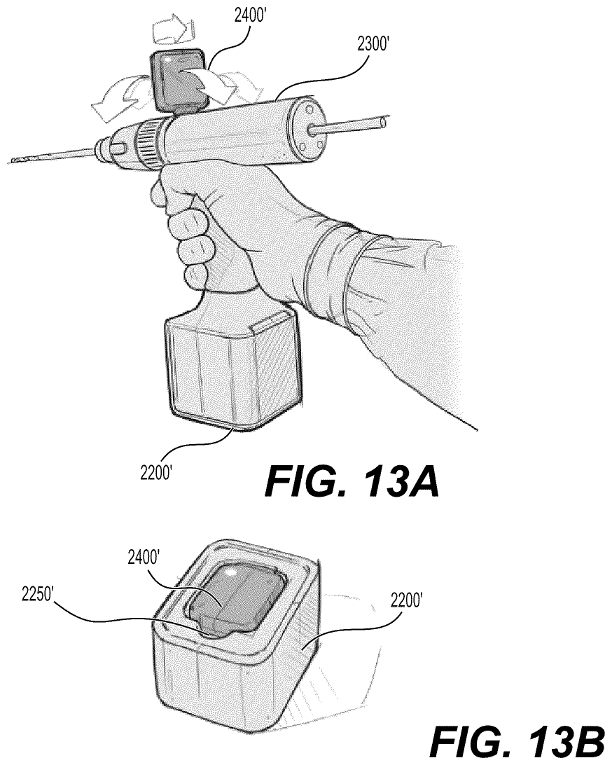

FIGS. 13A-13B depict yet another configuration of on-board external display 2400′ that is connected to a handpiece 2300′ (which is equivalent to the previously described handpiece 300). This example is like that of FIGS. 12A-12C but, rather than tethering the display 2400′ to the smart power supply housing 2200′ (which is equivalent to the previously described smart power supply housing 200) for power, the on-board display 2400′ can be removed from the handpiece 2300′ and charged within a recess 2250′ in the smart power supply housing 2200′.

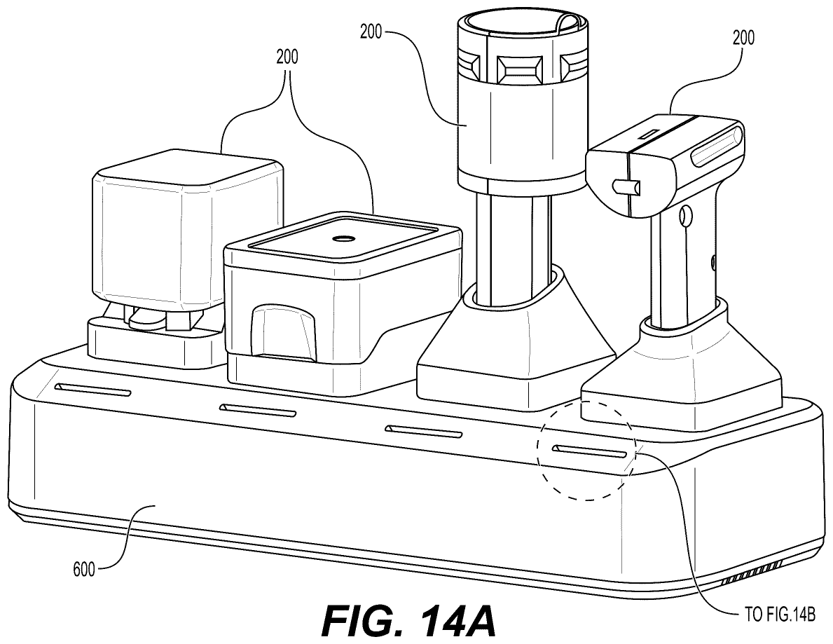

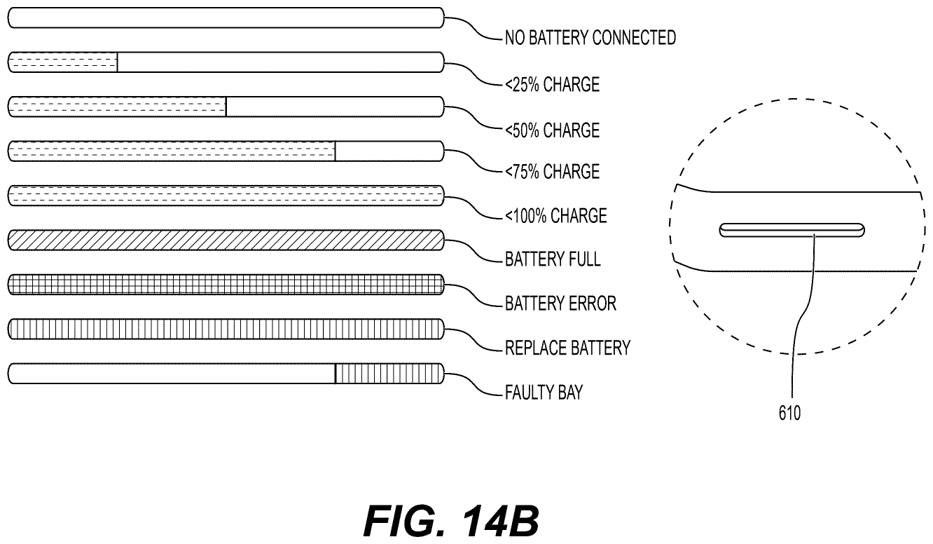

FIGS. 14A-14B depict a docking station 600 compatible with any of the previously described smart power supply housings (e.g., smart power supply housings 200, 1200, 2200, and 2200′) as well as a number of different orthopedic hand-held power tools (e.g., drills, saws, impact driver, and the like). The docking station 600 includes a plurality of digital dashboards 610, each associated with a respective docking bay. The digital dashboard 610, based on a charge state and/or a health state of the smart power supply housing 200, updates its screen to provide viewers useful feedback regarding the smart power supply housing 200. FIG. 14B depicts various exemplary screens presentable by the digital dashboard 610. It is noted that the cross-hatching is employed to denote exemplary colors the digital dashboard 610 can employ. Those skilled in the art will appreciate that, of course the visual presentation can be different than as shown without departing from the spirit and scope of the present disclosure. The upper six rows of exemplary screens depict what a viewer is presented with at various connection and charge states of the battery. If a battery error is detected by the docking station 600, the digital dashboard 610 is updated to reflect the battery error. If the docking station 600 detects that the battery needs replaced (e.g., the charge capacity of the smart power supply housing 200 has degraded below a predetermined limit), the digital dashboard 610 is updated to reflect that the battery needs to be replaced. If the docking station 600 detects that the docking bay is malfunctioning, the digital dashboard 610 is updated to reflect that, as seen in the bottommost row.

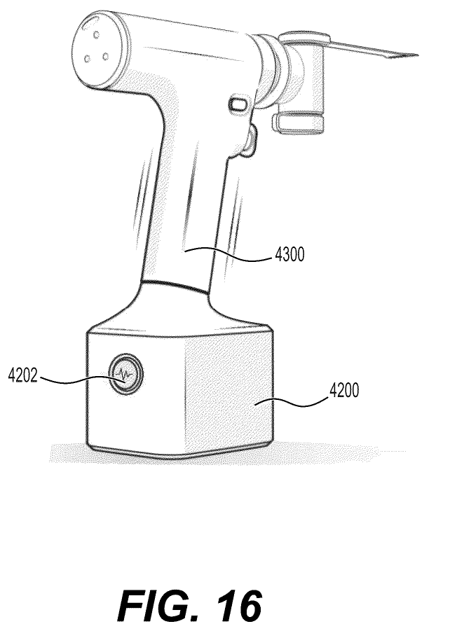

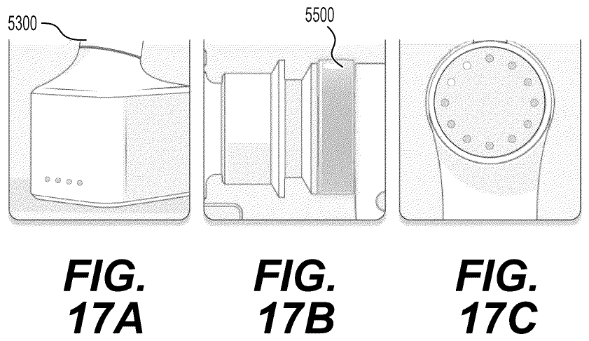

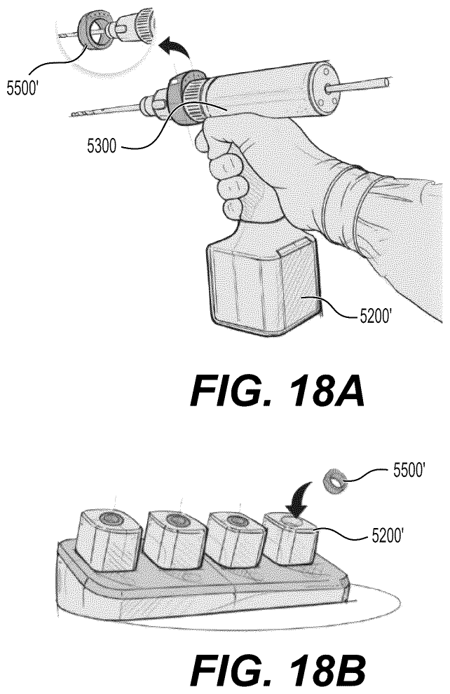

In addition, or as an alternative to the previously described external displays, the presently disclosed technology also can leverage other methods of relaying information to the users of the systems disclosed herein. For example, FIG. 15 depicts a handpiece 3300 (e.g., a reciprocating saw with a saw attachment 104) connected to a smart power supply housing 3200. The smart power supply housing 3200 can output audible sounds to convey information and alerts via a small speaker. FIG. 16 depicts a handpiece 4300 connected to a smart power supply housing 4200. The smart power supply housing 4200 or the handpiece 4300 can include haptics to deliver physical sensations to the holder to inform users of status changes, alerts, actions, and the like. An activation button 4202 can be included to control the activation and deactivation of this feature. FIGS. 17A-17C depict portions of another handpiece 5300 that employs various configurations of light indicators to provide feedback to the user. FIG. 17A, which is similar to the example depicted in FIG. 10, leverages discrete lights (e.g., light emitting diodes (LEDs)) on the smart power supply housing to communicate health, sync status, and various other alerts. FIG. 17B depicts a light pipe or ring 5500 that is made more focal to communicate health, sync status, and various other alerts. FIG. 17C leverages discrete lights (e.g., light emitting diodes (LEDs)) on the handpiece (in addition to, or alternative to, the lights of FIG. 17A) to communicate health, sync status, and various other alerts. FIGS. 18A-18B depict yet another exemplary handpiece 5300 that leverages light as a communication means. In this example, a removable light ring 5500′ is provided. As seen in FIG. 18B, the light ring 5500′ can be docked on the smart power supply housing 5200′ for charging, like the example of FIGS. 13A-13B.

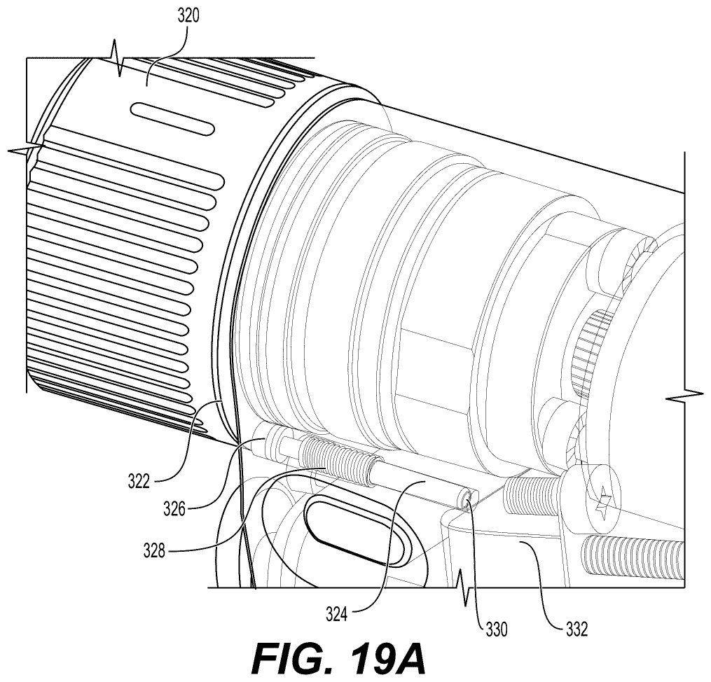

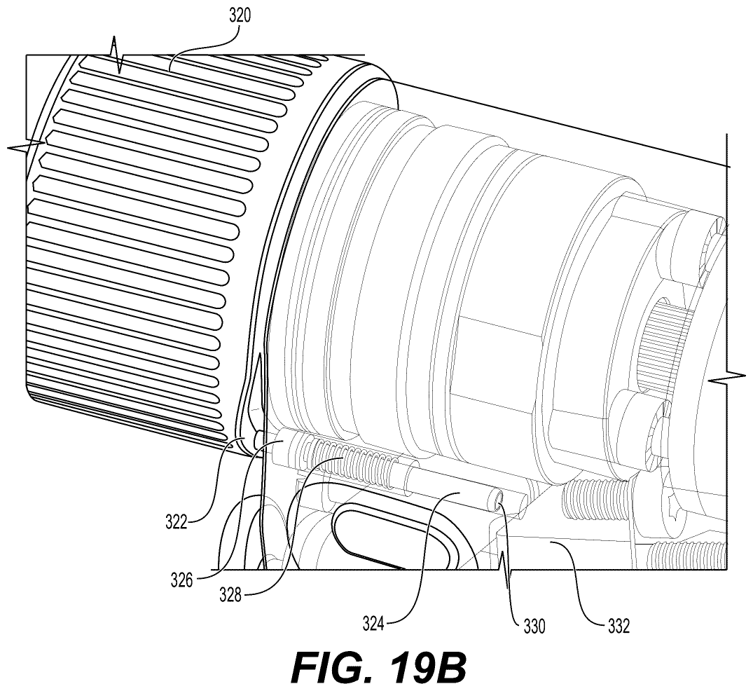

Reference is now made to FIGS. 19A-21. Some handpieces 300 in accordance with the present disclosure include speed selectors 320 that are rotated to change the speed and torque outputs of the tool head 100 (e.g., between a drill mode and a ream mode). However, being able to physically adjust these outputs directly on the tool head poses a problem when a specific screw type requires a torque range higher than the selected mode of the speed selector 320. Therefore, it is desirable to provide a sensing system that works in conjunction with the previously described features (e.g., the electronic screw finishing feature of FIGS. 6A-6B) to alert the user(s) in cases where the mode selected by the speed selector 320 would not supply sufficient output torque to insert the screws.

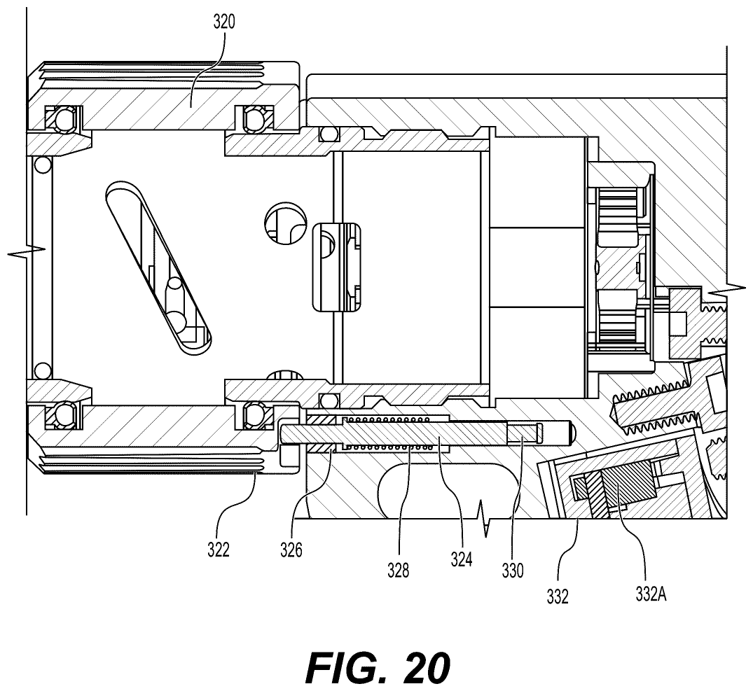

FIG. 19A depicts the handpiece 300 with the speed selector 320 in a first position or mode (e.g., in the ream mode), while FIG. 19B depicts the handpiece 300 with the speed selector 320 in a second position or mode (e.g., in the drill mode). The speed selector 320 is rotatable to move it between the first and second modes. It is noted that FIGS. 19A and 19B depict the handpiece 300 with portions of the body of the handpiece 300 removed to illustrate the relevant features of the presently described example. FIG. 20 is a cross-sectional view of the configuration of FIG. 19B.

As seen in FIG. 19B, the handpiece 300 includes a speed selector sensor 332A (provided in a control box 332) integrated within the body of the handpiece 300 that detects the set mode of the tool head 100. Moreover, the speed selector sensor 332A is in communication with the processor 225 of the smart power supply housing 200. The speed selector includes a cam surface 322 that engages a face of a plunger rod 324 that is biased towards the speed selector 320 by a spring 328. A seal 326 maintains a positioning of the plunger rod 324 and to prevent the spring 328 from becoming dislodged.

A magnet 330 is connected to an opposite face of the plunger rod 324 such that movement of the plunger rod 324 along the cam surface 322 causes translation of the plunger rod 324. In some examples, the speed selector sensor 332A includes a Hall sensor, such that translational movement of the magnet 330 (see FIGS. 19A to 19B, for example) causes a change in magnetic field/voltage in the Hall sensor. This change in voltage is indicative of the set mode of the speed selector 320.

The processor 225 of the smart power supply housing 200 is connected to the speed selector sensor 332A such that it receives the set mode signal detected by the speed selector sensor 332A and correlates the set mode to a torque output associated therewith. Thereafter, the processor 225 can compare the torque output of the set mode with an associated torque of the identified screw type (refer to the description of FIGS. 6A and 6B). Based on the comparison, the processor 225 can determine whether the torque output of the set mode is greater than or less than the associated torque of the identified screw type.

If the torque output of the set mode is less than the associated torque of the identified screw type, the processor 225 of the smart power supply housing 200 communicates with the processor 406 of the external display 400 such that the external display 400 displays a notification on the graphical user interface 402 that is indicative of the set mode of the speed selector being less than the associated torque of the identified screw type. In this way, the user(s) is alerted in instances where the set mode of the speed selector 320 is not capable of supplying the required output torque for a particular screw type.

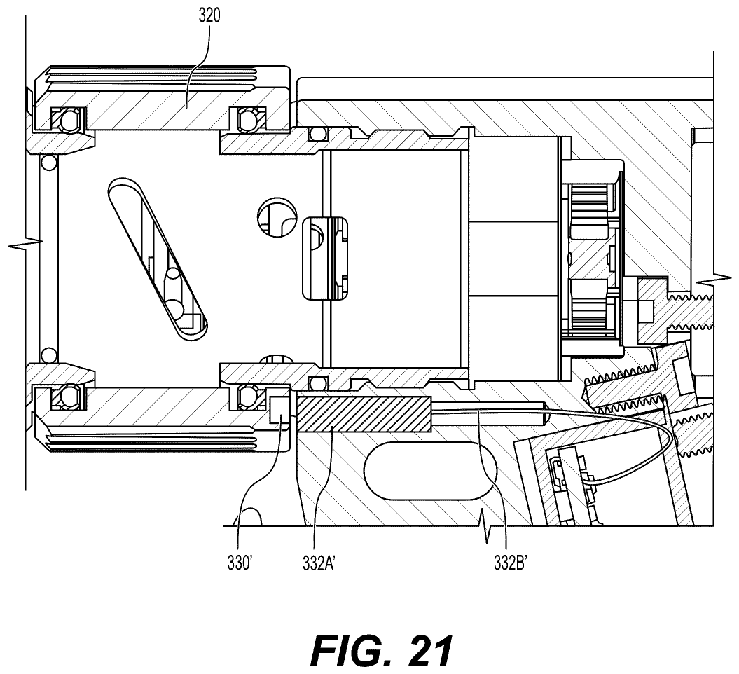

FIG. 21 illustrates a similar example to that of FIGS. 19A-20B. Therefore, only the features that differ from the previous example are discussed in relation to FIG. 21. In the example of FIG. 21, the magnet 330′ is directly connected (or rotationally fixed) to the speed selector 320′ such that movement of the speed selector 320′ (e.g., rotational movement) rotates the magnet 330′ relative to the Hall sensor 332A′ (which is connected to the control box 332′ via electrical interconnections 332B′).

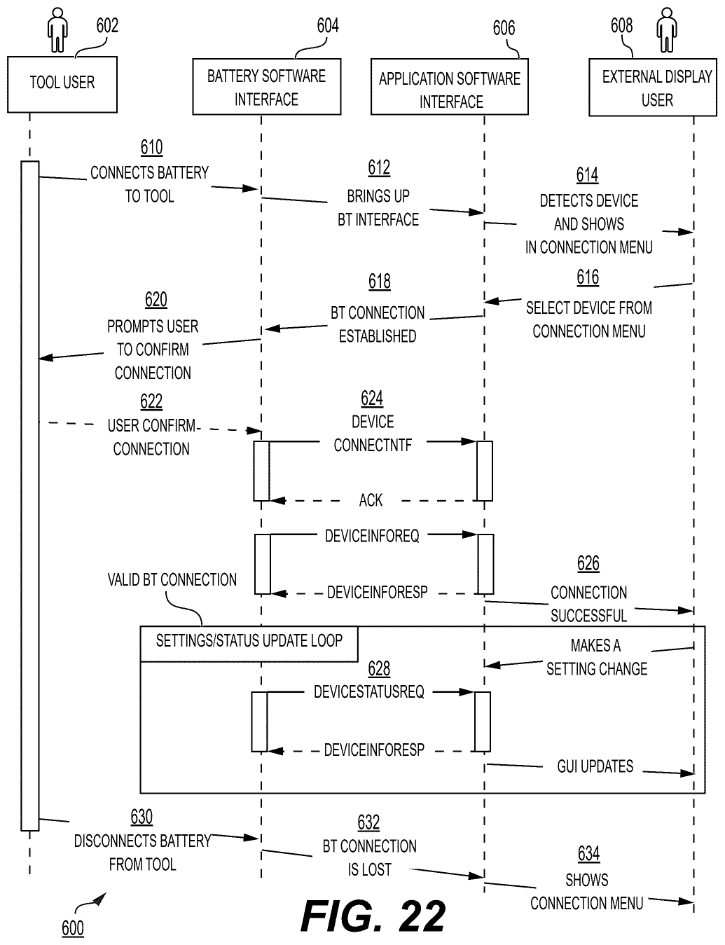

As discussed above, the smart power supply housing 200 and the display 400 comprise a wireless connection through which information (e.g., the parameters set on the display 400, the speed, torque, and/or position of the handpiece 300, etc.) is bi-directionally transmitted between the smart power supply housing 200 and the display 400. FIG. 22 shows an exemplary sequence diagram 600 for wireless communications between the smart power supply housing 200 and the display 400. Wireless communication in the present system generally requires four components: a tool user 602 (e.g., a surgeon holding the handpiece 300 powered by the smart power supply housing 200), a battery software interface 604 (e.g., the communications interface in the software running on the smart power supply housing, such as the communication interface module 245), application software interface 606 (e.g., the communications interface of the application software running on the external display 400, such as the communication interface module 408), and the external display user 608 (e.g., a user updating settings and viewing the status of the handpiece 300 on the external display 400 from outside of the sterile field). While it is noted that, in some examples, the external display user 608 and the tool user 602 can be the same user, the presently disclosed technology is particularly advantageous in scenarios where multiple users are required to use the handpiece 300 and adjust/view the settings associated therewith.

With continued reference to the flow diagram 600, in a first step, the tool user 602 connects 610 a smart power supply housing 200 to the handpiece 300. Connecting the smart power supply housing 200 prompts the battery software interface 604 to load 612 the wireless (e.g., Bluetooth; it is noted that the “BT” in flow diagram 600 refers to Bluetooth as an exemplary form of wireless communication) communication interface. The wireless communication interface is only loaded when it is connected to a handpiece 300 (i.e., it is otherwise not available to connect to an external display 400). The application software interface 606 detects 614 the handpiece 300 upon the connection 610 of the smart power supply housing 200 and makes it visible in a connection menu on the external display 400. The name of the connection is unique to the currently connected handpiece 300—for example, it could be the unique serial number of the handpiece 300.

The external display user 608 selects 616 the available connection to the handpiece 300 when it becomes available in the software. This selection indicates that the external display user 608 would like to wirelessly connect the external display 400 to the smart power supply housing 200 (and, therefore, also the connected handpiece 300). Selection of the handpiece establishes 618 a wireless connection with the smart power supply housing 200 solely for the purposes of finalizing the connection (i.e., no information regarding the handpiece's operating status, set parameters, sensor(s), etc. is available to the external display 400). A prompt is subsequently transmitted 620 to confirm the connection by the tool user 602. By doing so, both users 602 and 608 need to permit the connection between the handpiece 300 and the external display 400 to exchange information and for the external display 400 to function in the manner as described above.

Once the connection is confirmed 622, the smart power supply 200 transmits 626 a confirmation message to the application running on the external display 400 and the application requests 624 device information from the smart power supply housing 200. The device information contains the enabled smart features the connected handpiece 300 can support.

When the smart power supply housing 200 and external display 400 are fully connected to exchange information, the external display polls 628 the smart power supply housing 200 by sending a status request message to the smart power supply housing 200. The status request message contains the current enable state and configuration of any supported smart features. The status response message from the smart power supply housing 200 contains the error state of the smart power supply housing 200 and other real-time information that the processor 406 of the external display requires to update the graphical user interface 402 to give feedback to the external display user 608.

When the smart power supply housing 200 reports and error status to the external display 400, the processor 406 displays this error to the external display user 608. The external display user 608 needs to dismiss this error message to stop its display.

If the smart power supply housing 200 does not respond to successive status polling attempts from the application of the external display 400 or the wireless connection drops, the application prompts the external display user 608 to select another connection from the list of available connections.

When the smart power supply housing 200 is disconnected 630 from the handpiece 300, it will go into low power mode and drop 632 the wireless connection. In this case, the application of the external display 400 prompts 634 the external display user 608 to select another connection from the list of available connections.

It is noted that the smart power supply housing 200 is advantageously handpiece-agnostic. In other words, it is compatible across other handpieces (e.g., other types of drills, saws, impactors/impact drivers, etc.). Therefore, upon disconnecting 630 the smart power supply housing 200 from the previously mentioned handpiece 300, the handpiece user 602 can connect 610 the smart power supply housing 200 to another handpiece 300 (which may have different enabled smart features the new connected handpiece 300 can support compared with the previously connected handpiece 300), which restarts the sequence depicted in the flow diagram 600. Thus, the same smart power supply housing 200 can be swapped across handpieces 300 while still enabling all the smart features of the presently described system.

Aspects of the present disclosure are also provided by the following numbered Clauses:

Clause 1. A smart hand-held power tool system comprising: a handpiece (300) comprising a tool head (100) for performing an operation and at least one sensor (105); a smart power supply housing (200) connectable to the handpiece (300) and controlling at least one parameter setting or functionality associated with the operation of the handpiece, the smart power supply housing (200) comprising: a power supply (205) enclosed within the smart power supply housing (200); and housing circuitry (205) configured to: produce navigation, speed and/or torque outputs based on feedback data generated by the at least one sensor (105) associated with the handpiece (300), the smart power supply housing (200) and/or the tool head (100); and control at least one parameter setting associated with the operation of the handpiece; and a display (400) in communication with the smart power supply housing (200) and comprising: a graphical user interface (402); and display circuitry configured to display a plurality of interfaces on the graphical user interface, at least one interface of the plurality of pages comprising the at least one parameter setting.

Clause 2. The smart hand-held power tool system of clause 1, wherein the graphical user interface displays the produced navigation, speed and/or torque outputs.

Clause 3. The smart hand-held power tool system of any one of clauses 1-2, wherein the display is configured to (i) receive a selection of the at least one parameter setting and (ii) display the selection, and the housing circuitry is configured to control the at least one parameter setting in response to the selection.

Clause 4. The smart hand-held power tool system of clause 3, wherein the graphical user interface is configured to receive an input indicative of the selection of the at least one parameter setting.

Clause 5. The smart hand-held power tool system of clause 3 or clause 4, wherein the smart supply housing is configured to: receive an input indicative of the selection of the at least one parameter setting; and transmit the selection of the at least one parameter setting to the display for displaying the at least one parameter setting on the graphical user interface.

Clause 6. The smart hand-held power tool system of any one of clauses 1-5, wherein a first interface of the plurality of interfaces comprises a real-time guidance plot (452) comprising an indicator (452A) representative of an orientation trajectory (456) of the tool head relative to a target trajectory (454).

Clause 7. The smart hand-held power tool system of clause 6, wherein the real-time guidance plot is divided into quadrants.

Clause 8. The smart hand-held power tool system of any one of clauses 6-7, wherein the real-time guidance plot comprises a first target zone (452D), and the indicator comprises (i) a first appearance when a majority of the indicator is outside the first target zone and (ii) a second appearance when a majority of the indicator is within the first target zone.

Clause 9. The smart hand-held power tool system of clause 8, wherein the real-time guidance plot comprises a second target zone (452E), and the indicator comprises a third appearance when a majority of the indicator is inside the second target zone, the third appearance being indicative of the tool head being aligned, within a predetermined tolerance, with the target trajectory (454).

Clause 10. The smart hand-held power tool system of any one of clauses 6-9, wherein, in response to the orientation trajectory differing from the target trajectory more than a predetermined threshold, the housing supply circuitry or the display circuitry is configured to output an alert.

Clause 11. The smart hand-held power tool system of any one of clauses 6-10, further comprising a button (458, 1215B) operable to set the target trajectory.

Clause 12. The smart hand-held power tool system of clause 11, wherein the first interface comprises the button (458).

Clause 13. The smart hand-held power tool system of any one of clauses 11-12, wherein the smart power supply housing comprises the button (1215B).

Clause 14. The smart hand-held power tool system of any one of clauses 6-13, wherein the at least one parameter setting comprises the target trajectory.

Clause 15. The smart hand-held power tool system of any one of clauses 6-14, wherein the orientation trajectory comprises a first component and a second component.

Clause 16. The smart hand-held power tool system of any one of clauses 1-15, wherein the display circuitry is configured to identify a screw type to be used during the operation.

Clause 17. The smart hand-held power tool system of clause 16, wherein the display comprises a display sensor (442) that detects a feature (500) associated with the screw, and the feature comprises identifying information to identify the screw type.

Clause 18. The smart hand-held power tool system of clause 17, wherein the sensor comprises a camera, and the feature comprises a Quick Response (QR) code or a bar code.

Clause 19. The smart hand-held power tool system of any one of clauses 17-18, wherein the display sensor comprises a sensor antenna, and the feature comprises a feature antenna that communicates with the sensor antenna.

Clause 20. The smart hand-held power tool system of any one of clauses 17-19, wherein a second interface of the plurality of interfaces comprises a selectable button (444) configured to activate the display sensor.

Clause 21. The smart hand-held power tool system of clause 20, wherein the second interface comprises one or more regions (446) configured to display the identified screw type.

Clause 22. The smart hand-held power tool system of any one of clauses 20-21, wherein, in response to selection of the button, a sub-interface of the second interface is configured to be loaded on the graphical user interface, the sub-interface comprising a box region (448) for positioning the feature relative to the display sensor.

Clause 23. The smart hand-held power tool system of any one of clauses 20-22, wherein the second interface comprises a counter that is configured to count a number of screws used during the procedure.

Clause 24. The smart hand-held power tool system of any one of clauses 17-23, wherein the display circuitry is configured to register the feature detected by the display sensor in response to the display sensor detecting the feature for at least a predetermined period of time.

Clause 25. The smart hand-held power tool system of clause 24, wherein the predetermined period of time is one to two seconds.

Clause 26. The smart hand-held power tool system of any one of clauses 16-25, wherein (i) the housing circuitry or the display circuitry stores a database of screw types and associated torque values, (ii) the housing circuitry is configured to set the tool head to a torque value, within a predetermined tolerance, of the associated torque values based on the identified screw type, and (iii) the at least one parameter setting comprises the torque value.

Clause 27. The smart hand-held power tool system of any one of clauses 16-26, wherein the handpiece comprises: a speed selector (320) configured to set the tool head to a set mode of a plurality of modes, each mode comprising respective speed output and a respective torque output; a speed selector sensor (332A) (i) configured to detect the set mode of the tool head and (ii) in communication with the housing circuitry.

Clause 28. The smart hand-held power tool system of clause 27, wherein the housing circuitry is configured to: receive the set mode detected by the speed selector sensor; compare the torque output of the set mode with an associated torque of the identified screw type; and determine whether the torque output of the set mode is greater than or less than the associated torque of the identified screw type.

Clause 29. The smart hand-held power tool system of clause 28, wherein, in response to determining that the torque output of the set mode is less than the associated torque of the identified screw type, the display circuitry is configured to display a notification on the graphical user interface indicative of the set mode being less than the associated torque of the identified screw type.

Clause 30. The smart hand-held power tool system of any one of clauses 27-29, further comprising a magnet (330, 330′), wherein the speed selector sensor comprises a Hall sensor (332A, 332A′) configured to detect a change in magnetic field in response to the magnet moving relative to the Hall sensor.

Clause 31. The smart hand-held power tool system of clause 30, further comprising a plunger rod (324) connected to the magnet, wherein the speed selector comprises a cam surface configured to engage the plunger rod to move the magnet relative to the Hall sensor.

Clause 32. The smart hand-held power tool system of clause 30, wherein the magnet is directly connected to the speed selector such that movement of the speed selector moves the magnet relative to the Hall sensor.

Clause 33. The smart hand-held power tool system of any one of clauses 1-32, wherein the at least one sensor of the tool head is configured to detect an angular acceleration value of the handpiece.

Clause 34. The smart hand-held power tool system of clause 33, wherein a third interface (410) of the plurality of interfaces comprises a plurality of selectable sensitivity levels, each associated with a respective angular acceleration value or a respective angular velocity value, the at least one parameter setting comprising the respective angular acceleration value or the respective angular velocity value.

Clause 35. The smart hand-held power tool system of clause 34, wherein in response to selection of a sensitivity level of the plurality of sensitivity levels, the display circuitry is configured to transmit the associated angular acceleration value or the associated angular velocity value to the housing circuitry, and in response to the at least one sensor of the tool head detecting an angular acceleration value or an angular velocity value that is greater than the associated angular acceleration value or the associated angular velocity value, the housing circuitry is configured to stop power to the tool head.

Clause 36. The smart hand-held power tool system of any one of clauses 34-35, wherein the third interface or the smart power supply housing comprises a toggle (414) to selectively activate or deactivate a selected sensitivity level of the plurality of selectable sensitivity levels.

Clause 37. The smart hand-held power tool system of clause 36, wherein, in response to the toggle activating the selected sensitivity level, the display circuitry is configured to display an indicator (416) on the graphical user interface.

Clause 38. The smart hand-held power tool system of any one of clauses 36-37, wherein the smart power supply housing comprises at least one light, and, in response to the toggle activating the selected sensitivity level, the housing circuitry is configured to activate the at least one light.

Clause 39. The smart hand-held power tool system of any one of clauses 1-38, wherein the display is an off-board display detached from the handpiece.

Clause 40. The smart hand-held power tool system of clause 39, wherein the off-board display comprises one of a portable computing device, a desktop computing device, a base station computing device, or a screen.

Clause 41. The smart hand-held power tool system of any one of clauses 1-38, wherein the display is an on-board display that is detachably and/or adjustably connected to the handpiece.

Clause 42. The smart hand-held power tool system of clause 41, further comprising a ring (1310) rotatably mounted on the handpiece (1300), wherein the on-board display is removably connected to the ring.

Clause 43. The smart hand-held power tool system of clause 42, wherein the ring comprises a barrel (1312), and the on-board display is rotatably connected to the barrel.

Clause 44. The smart hand-held power tool system of any one of clauses 41-43, wherein the on-board display comprises three degrees of freedom.

Clause 45. The smart hand-held power tool system of clause 41, wherein the on-board display is tethered to the smart power supply housing via a cable (2410).

Clause 46. The smart hand-held power tool system of clause 45, wherein the cable provides power from the smart power supply housing to the on-board display.

Clause 47. The smart hand-held power tool system of any one of clauses 45-46, wherein the cable is routed along a groove (2300) in the handpiece.

Clause 48. The smart hand-held power tool system of any one of clauses 45-47, wherein the on-board display is connected to the handpiece via a hinge (2310).

Clause 49. The smart hand-held power tool system of clause 41, wherein the on-board display (2400′) is removable from the handpiece and chargeable within a recess (2250′) in the smart power supply housing (2200′).

Clause 50. The smart hand-held power tool system of any one of clauses 1-49, further comprising a docking station (600) for charging the smart power supply housing, wherein the docking station comprises a digital dashboard indicative of a charge state and a health state of the smart power supply housing.

Clause 51. The smart hand-held power tool system of any one of clauses 1-50, wherein the tool head comprises a drill bit (102).

Clause 52. The smart hand-held power tool system of any one of clauses 1-50, wherein the tool head comprises a saw (104).

Clause 53. The smart hand-held power tool system of any one of clauses 1-50, wherein the tool head comprises an impactor.

Clause 54. The smart hand-held power tool system of any one of clauses 1-53, wherein the smart power supply housing and the display comprise a wireless connection through which information is bi-directionally transmitted between the smart power supply housing and the display.

Clause 55. The smart hand-held power tool system of clause 54, wherein the information comprises the at least one parameter setting.

Clause 56. The smart hand-held power tool system of any one of clauses 1-53, wherein the handpiece is a first handpiece comprising a first tool head, the first tool head comprising a drill bit, the smart hand-held power tool system further comprises a second handpiece comprising a second tool head, the second tool head comprising a saw, and the smart power supply housing is connectable to the second handpiece and controls at least one parameter setting or functionality associated with the operation of the second handpiece.

Clause 57. A method of controlling handpieces for performing one or more operations, the method comprising: receiving a selection (616), to an external display from a first user, of a first handpiece from a connection menu, the first handpiece being available on the connection menu in response to connection of a smart power supply housing to the first handpiece, and the selection comprising a request to wirelessly connect the external display to the smart power supply housing; based on the selection of the first handpiece from the connection menu, transmitting (620) a prompt to the smart power supply housing to confirm the request to wirelessly connect the external display to the smart power supply housing; receiving (622) a confirmation, to the smart power supply housing from a second user, to wirelessly connect the external display to the smart power supply housing; and wirelessly connecting the external display to the smart power supply housing to permit information to be transmitted between the external display to the smart power supply housing, the information comprising one or smart features supported by the first handpiece.

Clause 58. The method of clause 57, wherein the first user is different from the second user.

Clause 59. The method of any one of clauses 57-58, further comprising: disconnecting (632) the external display from the smart power supply housing.

Clause 60. The method of clause 59, further comprising: receiving a selection, to the external display from the first user, of a second handpiece from the connection menu, the second handpiece being available on the connection menu in response to connection of the smart power supply housing to the second handpiece, and the selection comprising a request to wirelessly connect the external display to the smart power supply housing; based on the selection of the first handpiece from the connection menu, transmitting a prompt to the smart power supply housing to confirm the request to wirelessly connect the external display to the smart power supply housing; receiving a confirmation, to the smart power supply housing from the second user, to wirelessly connect the external display to the smart power supply housing; and wirelessly connecting the external display to the smart power supply housing to permit information to be transmitted between the external display to the smart power supply housing, the information comprising one or smart features supported by the second handpiece.

Clause 61. A smart hand-held power tool system comprising: a handpiece (300) including a tool head (100) performing an operation; a smart power supply housing (200) connectable to the handpiece (300) controlling at least one parameter setting and/or functionality associated with the operation of the smart hand-held power tool system; the smart power supply housing (200) including: a power supply (205) enclosed within the smart power supply housing (200); and a screen (210) associated with the smart power supply housing (200) displaying the at least one parameter setting and/or the functionality associated with the operation of the smart hand-held power tool system.

Clause 62. The system of clause 1, wherein the smart power supply housing (200) further comprises a graphical user interface module (235) disposed in the smart power supply housing (200) displaying and updating a graphical user interface on the screen (210), wherein the graphical user interface includes the at least one parameter setting and/or the functionality associated with the smart hand-held power tool system.

Clause 63. The system of any of clauses 61-62, wherein the at least one parameter setting and/or functionality associated with the operation of the tool head (100) displayed on the screen (210) includes at least one of: (i) a menu of available modes or functions: (ii) charge status of the power supply (205); (iii) wireless communication connection status; (iv) enabling/disabling anti-kickback functionality ceasing operation of the tool head in response to detected kickback; (v) adjusted and/or current maximum depth of insertion of the tool head (100); (vi) adjusted and/or current maximum operating speed of the tool head (100); (vii) warnings; and/or (viii) errors.

Clause 64. The system of any of clauses 61-63, further comprising at least one sensor (105) generating feedback data; wherein the at least one sensor (105) is associated with the handpiece (300), the smart power supply housing (200) or the tool head (100).

Clause 65. The system of clause 64, wherein the at least one sensor (105) is: an accelerometer; a gyroscope; an optical imaging sensor; an electromagnet and/or a magnetometer.

Clause 66. The system of clause 64, wherein the smart power supply housing (200 further comprises an inertial measurement unit (255) generating navigation, acceleration and/or torque measurements based on the feedback data detected by the at least one sensor (105).

Clause 67. The system of clause 66, wherein the screen (210) displays the generated navigation, acceleration and/or torque measurements.

Clause 68. A method of using a smart hand-held power tool system including: a handpiece (300) including a tool head (100) performing an operation; a smart power supply housing (200) connectable to the handpiece (300) controlling at least one parameter setting and/or functionality associated with the operation of the smart hand-held power tool system; the smart power supply housing (200) including: a power supply (205) enclosed within the smart power supply housing (200); and a screen (210) associated with the smart power supply housing (200) displaying the at least one parameter setting and/or the functionality associated with the operation of the smart hand-held power tool system; the method comprising the step of: controlling the at least one parameter setting and/or the functionality associated with the operation of the smart hand-held power tool system via the smart power supply housing (200).

Clause 69. The method of clause 68, wherein the smart power supply housing (200) further comprises: a graphical user interface module (235) disposed in the smart power supply housing (200) displaying and updating a graphical user interface on the screen (210), wherein the graphical user interface includes the at least one parameter setting and/or the functionality associated with the operation of the smart hand-held power tool system.

Clause 70. The method of any of clauses 68-69, wherein the at least one parameter setting and/or functionality associated with the operation of the tool head (100) displayed on the screen (210) includes at least one of: (i) a menu of available modes or functions: (ii) charge status of the power supply (205); (iii) wireless communication connection status; (iv) enabling/disabling anti-kickback functionality ceasing operation of the tool head in response to detected kickback; (v) adjusted and/or current maximum depth of insertion of the tool head (100); (vi) adjusted and/or current maximum operating speed of the tool head (100); (vii) warnings; and/or (viii) errors.

Clause 71. The method of any of Clauses 68-70, further comprising at least one sensor (105) detecting feedback data; wherein the at least one sensor (105) is associated with the handpiece (300), the smart power supply housing (200) or the tool head (100).

Clause 72. The method of clause 71, wherein the at least one sensor (105) is: an accelerometer; a gyroscope; an optical imaging sensor; an electromagnet and/or a magnetometer.

Clause 73. The method of any of clauses 71-72, wherein the smart power supply housing (200) further comprises an inertial measurement unit (255) generating navigation, acceleration and/or torque measurements based on the feedback data detected by the at least one sensor (105).

Clause 74. The method of Clause 73, wherein the screen (210) displays the generated navigation, acceleration and/or torque measurements.

Clause 75. A smart hand-held power tool system comprising: a handpiece (300) operating a tool head (100); a smart power supply housing (200) connectable to the handpiece (300); the smart power supply housing (200) including: a power supply (205) enclosed within the smart power supply housing (200); and an inertial measurement unit (255) disposed in the smart power supply housing (200) producing navigation, acceleration and/or torque measurements based on feedback data generated by at least one sensor (105) associated with the handpiece (300), the smart power supply housing (200) and/or the tool head (100).

Clause 76. The system of Clause 75, wherein the at least one sensor (105) is: an accelerometer; a gyroscope; an optical imaging sensor; an electromagnet and/or a magnetometer.

The descriptions contained herein are examples of embodiments of the present disclosure and are not intended in any way to limit the scope of the invention. As described herein, the present disclosure contemplates many variations and modifications of a smart (i.e., intelligent) hand-held power tool system in which parameter settings and/or functionality associated with the smart (i.e., intelligent) hand-held power tool system are controllable and displayed on a screen/monitor of the smart (i.e., intelligent) power supply housing. Such functionality performed by the smart (i.e., intelligent) power supply housing may include advanced/enhanced assistant features (e.g., navigation, geofencing, current insertion depth and/or anti-kickback using acceleration and/or torque measurements generated by an Inertial Measurement Unit based on feedback data detected by the sensor(s)). Modifications and variations apparent to those having skilled in the pertinent art according to the teachings of this disclosure are intended to be within the scope of the claims which follow.

What is claimed is:

1. A smart hand-held power tool system comprising:
a handpiece comprising a tool head for performing an operation and at least one sensor;
a smart power supply housing connectable to the handpiece and controlling at least one parameter setting or functionality associated with the operation of the handpiece, the smart power supply housing comprising:
a power supply enclosed within the smart power supply housing; and
housing circuitry configured to:
produce navigation, speed and/or torque outputs based on feedback data generated by the at least one sensor associated with the handpiece, the smart power supply housing and/or the tool head; and
control at least one parameter setting associated with the operation of the handpiece; and
a display in communication with the smart power supply housing and comprising:
a graphical user interface; and
display circuitry configured to display a plurality of interfaces on the graphical user interface, at least one interface of the plurality of interfaces comprising the at least one parameter setting.

2. The smart hand-held power tool system of claim 1, wherein the graphical user interface displays the produced navigation, speed and/or torque outputs.

3. The smart hand-held power tool system of claim 1, wherein the display is configured to (i) receive a selection of the at least one parameter setting and (ii) display the selection, and the housing circuitry is configured to control the at least one parameter setting in response to the selection.

4. The smart hand-held power tool system of claim 3, wherein the smart power supply housing is configured to:
receive an input indicative of the selection of the at least one parameter setting; and
transmit the selection of the at least one parameter setting to the display for displaying the at least one parameter setting on the graphical user interface.

5. The smart hand-held power tool system of claim 1, wherein a first interface of the plurality of interfaces comprises a real-time guidance plot comprising an indicator representative of an orientation trajectory of the tool head relative to a target trajectory.

6. The smart hand-held power tool system of claim 5, wherein
the real-time guidance plot comprises a first target zone, and
the indicator comprises (i) a first appearance when a majority of the indicator is outside the first target zone and (ii) a second appearance when a majority of the indicator is within the first target zone.

7. The smart hand-held power tool system of claim 1, wherein the display circuitry is configured to identify a screw type to be used during the operation.

8. The smart hand-held power tool system of claim 7, wherein the display comprises a display sensor that detects a feature associated with the screw, and the feature comprises identifying information to identify the screw type.

9. The smart hand-held power tool system of claim 7, wherein (i) the housing circuitry or the display circuitry stores a database of screw types and associated torque values, (ii) the housing circuitry is configured to set the tool head to a torque value, within a predetermined tolerance, of the associated torque values based on the identified screw type, and (iii) the at least one parameter setting comprises the torque value.

10. The smart hand-held power tool system of claim 7, wherein the handpiece comprises:
a speed selector configured to set the tool head to a set mode of a plurality of modes, each mode comprising respective speed output and a respective torque output;
a speed selector sensor (i) configured to detect the set mode of the tool head and (ii) in communication with the housing circuitry.

11. The smart hand-held power tool system of claim 1, wherein the at least one sensor of the tool head is configured to detect an angular acceleration value of the handpiece.

12. The smart hand-held power tool system of claim 11, wherein a third interface of the plurality of interfaces comprises a plurality of selectable sensitivity levels, each associated with a respective angular acceleration value or a respective angular velocity value, the at least one parameter setting comprising the respective angular acceleration value or the respective angular velocity value.

13. The smart hand-held power tool system of claim 12, wherein
in response to selection of a sensitivity level of the plurality of selectable sensitivity levels, the display circuitry is configured to transmit the associated angular acceleration value or the associated angular velocity value to the housing circuitry, and
in response to the at least one sensor of the tool head detecting an angular acceleration value or an angular velocity value that is greater than the associated angular acceleration value or the associated angular velocity value, the housing circuitry is configured to stop power to the tool head.

14. The smart hand-held power tool system of claim 1, wherein the display is an off-board display detached from the handpiece.

15. The smart hand-held power tool system of claim 1, wherein the display is an on-board display that is detachably and/or adjustably connected to the handpiece.

16. The smart hand-held power tool system of claim 1, further comprising a docking station for charging the smart power supply housing, wherein the docking station comprises a digital dashboard indicative of a charge state and a health state of the smart power supply housing.

17. The smart hand-held power tool system of any claim 1, wherein the smart power supply housing and the display comprise a wireless connection through which information is bi-directionally transmitted between the smart power supply housing and the display.

18. The smart hand-held power tool system of claim 1, wherein
the handpiece is a first handpiece comprising a first tool head, the first tool head comprising a drill bit,
the smart hand-held power tool system further comprises a second handpiece comprising a second tool head, the second tool head comprising a saw, and
the smart power supply housing is connectable to the second handpiece and controls at least one parameter setting or functionality associated with the operation of the second handpiece.

19. A smart hand-held power tool system comprising:
a handpiece including a tool head performing an operation;
a smart power supply housing connectable to the handpiece controlling at least one parameter setting or functionality associated with the operation of the smart hand-held power tool system; the smart power supply housing including:
a power supply enclosed within the smart power supply housing; and
a screen associated with the smart power supply housing displaying the at least one parameter setting or the functionality associated with the operation of the smart hand-held power tool system.

20. A smart hand-held power tool system comprising:
a handpiece operating a tool head;
a smart power supply housing connectable to the handpiece;
the smart power supply housing including: a power supply enclosed within the smart power supply housing; and
an inertial measurement unit disposed in the smart power supply housing producing navigation, acceleration or torque measurements based on feedback data generated by at least one sensor associated with the handpiece, the smart power supply housing or the tool head.
